(19) United States
(12) Patent Application Publication
(10) Pub. No.: US 2024/0105341 A1
(43) Pub. Date: Mar. 28, 2024

(54) SYSTEMS AND METHODS FOR ESTIMATING TUMOR GROWTH

(71) Applicant: AMGEN INC., Thousand Oaks, CA (US)

(72) Inventors: Arya POURZANJANI, Davis, CA (US); Khamir MEHTA, Danville, CA (US); Vijay Vishesh UPRETI, South San Francisco, CA (US)

(21) Appl. No.: 18/371,318

(22) Filed: Sep. 21, 2023

Related U.S. Application Data
(60) Provisional application No. 63/408,885, filed on Sep. 22, 2022.

(51) Int. Cl. G16H 50/30; G16H 10/60; G16H 50/70

(52) U.S. Cl. CPC G16H 50/30; G16H 10/60; G16H 50/70

(57) ABSTRACT

A method for estimating tumor growth dynamics includes obtaining, by one or more processors, progression-free survival (PFS) data for a plurality of patients, the PFS data indicating (i) a plurality of observation times, and (ii) how many of the plurality of patients, at each of the plurality of observation times, had a PFS event within a most recent time window; determining, by the one or more processors and based on the PFS data, a population distribution for one or more patient-specific parameters; obtaining, by the one or more processors, measured tumor growth data for a particular patient subject to a drug treatment; estimating, by the one or more processors, tumor growth for the particular patient based on (i) the measured tumor growth data and (ii) the population distribution for the one or more patient-specific parameters; and causing, by the one or more processors, a display to present a visual indication of the estimated tumor growth for the particular patient.

### CROSS REFERENCE TO RELATED APPLICATIONS

The present application claims the benefit of U.S. Provisional Patent Application Ser. No. 63/408,885 filed on Sep. 22, 2022, and entitled “SYSTEMS AND METHODS FOR ESTIMATING TUMOR GROWTH,” which is herein incorporated by reference in its entirety.

### FIELD OF THE DISCLOSURE

The present application relates generally to tumor growth analysis for oncology patients, and more specifically to the modeling and estimation of contributions to tumor growth rates.

### BACKGROUND

Tumor growth analysis is essential for experimental oncology research. Anticancer treatments work through decreasing tumor growth over time by shrinking the tumor and/or slowing growth, resulting in an improvement of patient's symptoms and prolongation of overall survival. Progression-free survival (PFS) can be the length of time during and after the treatment of a disease that a patient lives with the disease but it does not get worse. The PFS can be predictive of the overall survival.

### BRIEF SUMMARY

In one aspect, a computer-implemented method for estimating tumor growth comprises obtaining, by one or more processors, progression-free survival (PFS) data for a plurality of patients, the PFS data indicating (i) a plurality of observation times, and (ii) how many of the plurality of patients, at each of the plurality of observation times, had a PFS event within a most recent time window; determining, by the one or more processors and based on the PFS data, a population distribution for one or more patient-specific parameters; obtaining, by the one or more processors, measured tumor growth data for a particular patient subject to a drug treatment; estimating, by the one or more processors, tumor growth for the particular patient based on (i) the measured tumor growth data and (ii) the population distribution for the one or more patient-specific parameters; and causing, by the one or more processors, a display to present a visual indication of the estimated tumor growth for the particular patient.

In some embodiments, determining the population distribution for the one or more patient-specific parameters comprise determining a growth curve function comprising the one or more patient-specific parameters. In some embodiments, the growth curve function comprises at least one of an exponential growth function, a logistic growth function, or an ordinary differential function. In some embodiments, the one or more patient-specific parameters comprise a parameter for baseline-normalized sum-of-longest diameters (SLD) measurement. In some embodiments, the one or more patient-specific parameters comprise a parameter for growth rate.

In some embodiments, the parameter for growth rate comprises a parameter for baseline growth rate without treatment. In some embodiments, the one or more patient-specific parameters comprise a parameter for a proportion of drug-sensitive tumor cells in a patient of the plurality of patients. In some embodiments, a value of the parameter for the proportion of drug-sensitive tumor cells in the patient of the plurality of patients ranges from 0 to 1. In some embodiments, the growth curve function is time-dependent. In some embodiments, the determining the population distribution for the one or more patient-specific parameters includes fitting the growth curve function to observations at the plurality of observation times.

In some embodiments, the growth curve function is a logistic growth function; and the one or more patient-specific parameters include a plurality of parameters of the logistic growth function. In some embodiments, estimating tumor growth for the particular patient includes: modeling a tumor growth rate for the particular patient using a pharmacokinetic-pharmacodynamic (PKPD) model having a first term representing tumor size variation in the particular patient without the drug treatment and a second term representing a contribution to tumor size variation in the particular patient due to the drug treatment; and jointly estimating one or more parameters of the first term and one or more parameters of the second term by fitting the PKPD model to the measured tumor growth data, in part by using the population distribution for the one or more patient-specific parameters to set constraints for the one or more parameters of the first term.

In some embodiments, the one or more parameters of the second term include one or more of: a drug concentration in plasma of the particular patient; a max kill rate for the particular patient; and half-maximal effective concentration (EC50). In some embodiments, estimating the tumor growth further comprises obtaining an overall response rate for the particular patient subject to the drug treatment. In some embodiments, estimating the tumor growth further comprises obtaining one or more nontarget events for the particular patient subject to the drug treatment.

In some embodiments, the observations include, for each patient of the plurality of patients, a first observation at a first time indicating that the patient did not have a PFS event before the first time, and a second observation at a second time indicating that the patient had a PFS event before the second time. In some embodiments, the PFS data corresponds to a specific cancer type; and estimating tumor growth for the particular patient includes estimating tumor growth for a patient diagnosed with the specific cancer type. In some embodiments, causing the display to present the visual indication of the estimated tumor growth for the particular patient includes causing the display to show a trajectory of tumor growth for the particular patient if the particular patient had not had the drug treatment.

In some embodiments, the population distribution for the one or more patient-specific parameters includes a log-normal distribution. In some embodiments, the PFS data comprises at least one of a digitized PFS plot or a PFS risk table. In some embodiments, the PFS data indicates how many of the plurality of patients, at each of the plurality of observation times and within the most recent time window, had a baseline-normalized sum-of-longest diameters (SLD) measurement of at least 1.2 or had new lesions appear. In some embodiments, the plurality of patients represented by the PFS data are patients known to be associated with ineffective drug treatments. In some embodiments, the method further comprises adjusting a dose of the drug treatment based on the estimated tumor growth for the particular patient.

In another aspect, a computer system for estimating tumor growth, the computer system comprising: a data storage device storing processor-readable instructions; and a processor configured to execute the instructions to perform a method including: obtaining, by one or more processors, progression-free survival (PFS) data for a plurality of patients, the PFS data indicating (i) a plurality of observation times, and (ii) how many of the plurality of patients, at each of the plurality of observation times, had a PFS event within a most recent time window; determining, by the one or more processors and based on the PFS data, a population distribution for one or more patient-specific parameters; obtaining, by the one or more processors, measured tumor growth data for a particular patient subject to a drug treatment; estimating, by the one or more processors, tumor growth for the particular patient based on (i) the measured tumor growth data and (ii) the population distribution for the one or more patient-specific parameters; and causing, by the one or more processors, a display to present a visual indication of the estimated tumor growth for the particular patient.

In yet another aspect, a non-transitory computer-readable medium containing instructions for estimating tumor growth that, when executed by a processor, cause the processor to perform a method comprising: obtaining, by one or more processors, progression-free survival (PFS) data for a plurality of patients, the PFS data indicating (i) a plurality of observation times, and (ii) how many of the plurality of patients, at each of the plurality of observation times, had a PFS event within a most recent time window; determining, by the one or more processors and based on the PFS data, a population distribution for one or more patient-specific parameters; obtaining, by the one or more processors, measured tumor growth data for a particular patient subject to a drug treatment; estimating, by the one or more processors, tumor growth for the particular patient based on (i) the measured tumor growth data and (ii) the population distribution for the one or more patient-specific parameters; and causing, by the one or more processors, a display to present a visual indication of the estimated tumor growth for the particular patient.

### BRIEF DESCRIPTION OF THE DRAWINGS

The skilled artisan will understand that the figures, described herein, are included for purposes of illustration and are not limiting on the present disclosure. The drawings are not necessarily to scale, emphasis instead being placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 is a simplified block diagram of an example system that can implement the tumor growth modeling and estimation techniques disclosed herein.

FIG. 2 depicts an example progression-free survival (PFS) plot with an associated risk table, in accordance with some embodiments of the technology described herein.

FIG. 3 is a plot depicting an example selection of a single, fixed PFS event time for a particular patient within a particular PFS window and the associated exponential growth curve, in accordance with some embodiments of the technology described herein.

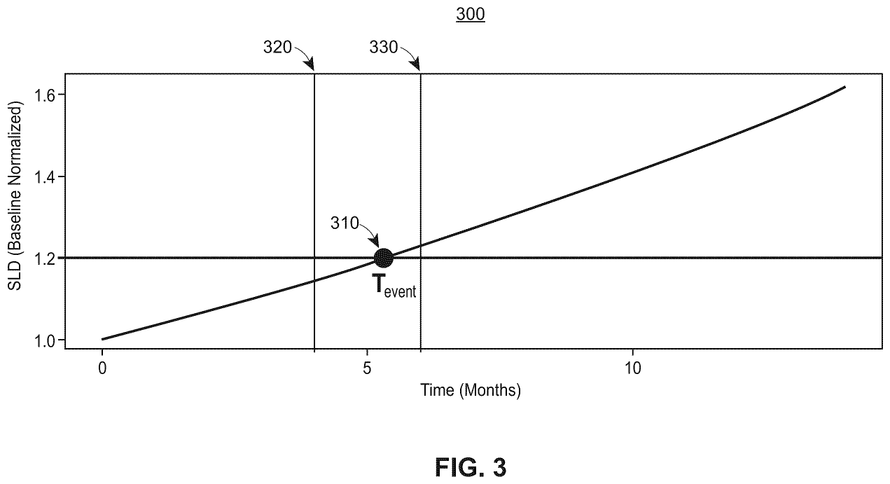

FIG. 4 is a plot depicting three exemplary possible PFS event times for a particular patient within a particular PFS window, and the associated exponential growth curves, in accordance with some embodiments of the technology described herein.

FIG. 5 is a plot depicting noise in SLD measurements over time for an example patient, in accordance with some embodiments of the technology described herein.

FIG. 6 depicts measured tumor doubling times across populations of patients, for each of a variety of cancer types, in accordance with some embodiments of the technology described herein.

FIG. 7 depicts estimated drug concentrations in plasma (top row) and estimated tumor growth (bottom row) for three example combinations of drug dose and cancer type, with estimated tumor growth showing both tumor growth with the drug and tumor growth without the drug, in accordance with some embodiments of the technology described herein.

FIG. 8 is a flow diagram of an example method for estimating tumor growth, in accordance with some embodiments of the technology described herein.

FIG. 9 depicts an exemplary generative process of the data, in accordance with some embodiments of the technology described herein.

FIG. 10 depicts one or more exemplary posterior draws of SLD for a single patient from the control arm who progressed at the second post-baseline scan with the ground truth values overlayed in white, in accordance with some embodiments of the technology described herein.

FIG. 11 depicts exemplary marginal posterior values for each patient estimated from the model along with ground truth values, in accordance with some embodiments of the technology described herein.

FIG. 12 shows exemplary posterior draws of the trial and arm-specific parameters with their ground truth values overlayed, in accordance with some embodiments of the technology described herein.

FIG. 13 depicts exemplary plots associated with a PFS curve and total number of responses for each draw that is overlayed with observed values in a posterior predictive check (PPC) to assess the adequacy of the fits, in accordance with some embodiments of the technology described herein.

FIG. 14 shows the simulated number of responses and PFS curves for the four arms of the simulated trial, in accordance with some embodiments of the technology described herein.

FIG. 15 is a schematic diagram of an illustrative computing device with which aspects described herein may be implemented.

### DETAILED DESCRIPTION

To assess the efficacy of a drug for oncology patients (e.g., during early-stage drug trials), observations of tumor/lesion size can be gathered over a time period during which the drug is administered. While tumor growth rate measurements are helpful, they may be insufficient because tumor growth rates can depend on two competing factors. One factor can be the rate at which a tumor would grow in the absence of the drug treatment, and the other factor can be the rate at which the administered drug is shrinking the tumor. Unless the individual contributions of each of these factors can be accurately estimated, it can be difficult to assess whether the drug is effective at all, much less determine the best dosing regimen, or determine which patient population can benefit most from the drug.

A technique has been proposed (referred to herein as the “Kay technique”) to isolate and assess the tumor growth rate in the absence of the drug treatment in which data from progression-free survival (PFS) plots is used to estimate tumor size doubling times per the “sum-of-longest diameters” (SLD) of target lesions metric. See Kay et al., The AAPS Journal, 21:27, Estimation of Solid Tumor Doubling Times from Progression Free Survival Plots Using a Novel Statistical Approach (2019). However, the Kay technique does not accurately account for particular uncertainties in the PFS plots, such as uncertainty regarding where a PFS event occurred within a given time window, and uncertainty due to SLD measurement error/noise. Moreover, the Kay technique rigidly applies certain assumptions (e.g., exponential tumor growth).

Some techniques and modeling methods can describe sum-of-longest diameter (SLD) measurements and their continuous response over time to some drug treatment administered, however, data is usually not publicly available for these techniques and modeling methods. Furthermore, some approaches can extract certain publicly available data and assume exponential growth due to their model assumptions, but the extracted publicly available data may not be informative of underlying tumor dynamics. Thus, there remains a need for tumor growth modeling that can be both more accurate and more flexible.

To address the aforementioned problems pertaining to tumor growth modeling, systems and methods disclosed herein can provide a more flexible modeling approach to more accurately learn one or more parameters (e.g., patient-specific parameters associated with tumor growth). Systems and methods disclosed herein can describe longitudinal sum-of-longest diameter (SLD) measurements and their continuous response over time to some drug treatment administered in a specific longitudinal regimen. This can yield several advantages over traditional method which typically considers a snapshot of time. First, systems and methods disclosed herein can capture the effect on response that may happen when exposure varies over time, which happens when patients have, for example, dose reductions, varied dosing regimens, or exposure of lowering anti-drug antibodies. Second, because the entire longitudinal history of SLD is modelled, multiple quantities of interest such as the maximum reduction, the progression time, the time to maximum reduction, or the doubling time can be captured by one unified model. Systems and methods disclosed herein can incorporate non-target progression events as time-to-event data modelled by a hazard function, this can be taken a step further and common endpoints such PFS and overall response rate (or ORR) can be captured. Third, because of its semi-mechanistic nature, one or more parameters can have mechanistic interpretations which allows for a rich set of possibilities for simulation and facilitates generalization to new regimes. For example, estimates of certain parameters such as kinetic constants (e.g., killing rate) can be borrowed or extrapolated from preclinical data or published studies to compare treatments under similar conditions or estimate the efficacy of novel treatments.

Systems and methods disclosed herein can also address: how early phase 1 results can generalize to earlier lines of therapy by using one or more parameters (e.g., patient-specific parameters or non-patient-specific parameters) to capture the dynamics of groups of patients with different lines of therapy; how a drug can perform head-to-head against an existing standard of care by using one or more parameters to capture drug-specific effects; how a drug can perform in combination with an existing standard of care by combining one or more parameters to simulate the combinations; and how many patients may be recruited in a trial to demonstrate these results in a statistically significant way by simulating entire trials with the systems and methods disclosed herein to account for variation at all levels.

While individual SLD data is usually not publicly available, endpoints such as PFS and ORR, which are directly derived from individual data, are often published. Because the relationship between such published data and the underlying individual SLD data is causal and unidirectional in nature, following a specific set of rules, publicly available endpoints such as PFS and ORR can be utilized to infer parameters in the systems and methods disclosed herein. In some situations, certain parameters can be estimated from individual SLD data from external datasets. One or more parameters that capture the dynamics of a cohort with earlier lines of therapy can be learned from data from those cohorts not available in phase 1. Tumor killing rates for other drugs can be learned from data of patients taking that drug.

Systems and methods disclosed herein can use Bayesian generative models, or probabilistic graphical models, to provide a very flexible and general framework for modelling how a set of observed data may have arisen from a set of underlying causes. Such models can be represented by probabilistic diagrams that encode the joint probability distribution between unobserved variables and observed data and can be used commonly in the Bayesian statistics and machine learning methods. This joint distribution can be subsequently used to infer causal relationships and parameters of interest by conditioning on observed data and applying Bayes' rule. This inference process of Bayesian inversion can be made particularly easy and flexible recently with the advent of several probabilistic programming languages such as BUGS, JAGS, Stan, PyMC3, Turing, and Pyro. The advantages of using Bayesian generative models can include specifying that the data generating process (conditional distributions) is natural and can provide the joint distribution; specifying the model in a probabilistic programming language (PPL) by simply coding the generative process because Bayesian inference can comprise the joint distribution; and implementing any complicated distributions, numerical methods, or constraints.

The systems and methods disclosed herein using Bayesian generative models can estimate general tumor dynamic information from published studies containing PFS and ORR information. Uncertainties in PFS data can be better accounted for by directly modeling tumor growth from information in PFS plots and/or risk tables, rather than randomly selecting a fixed event time for each patient (e.g., as in the Kay technique). In particular, the systems and methods disclosed herein can use the start and end points of time windows as censored observations for that particular patients have not yet had or have had, respectively, the event of baseline-normalized SLD reaching 1.2. By not artificially constraining the event time for each patient to a specific, fixed time, the overall distribution for the patient population reflected by the PFS data can more accurately reflect uncertainties, including uncertainty in the PFS event time within a given window as well as uncertainty due to SLD measurement error or noise. Moreover, the systems and methods disclosed herein may not assume a particular type of growth (e.g., exponential growth). The systems and methods disclosed herein can also be used to jointly estimate tumor growth parameters alongside with pharmacokinetic-pharmacodynamic (PKPD) parameters of a PKPD model. This can include parameters that indicate the contributions of both baseline (treatment-free) tumor growth and drug-related tumor shrinkage to the overall tumor growth rate which would otherwise be difficult to estimate without the use of supplemental and/or internal PFS data. The systems and methods disclosed herein can also be used to supplement existing non-public studies.

The system and methods disclosed herein can provide a deeper, more accurate understanding of tumor growth contributions in patients, across different indications (e.g., billary cancer, pancreatic cancer, testicular cancer, etc.). Moreover, the system and methods disclosed herein can allow the effects of a drug to be modeled without running a control group not subject to the drug treatment. This in turn can greatly reduce the amount of time, money, and/or other resources that can be expended before making important decisions such as whether to advance a drug study to a next stage (e.g., whether to expand a drug trial to a larger patient population).

The various concepts introduced above and discussed in greater detail below may be implemented in any of numerous ways, and the described concepts are not limited to any particular manner of implementation. Examples of implementations are provided for illustrative purposes.

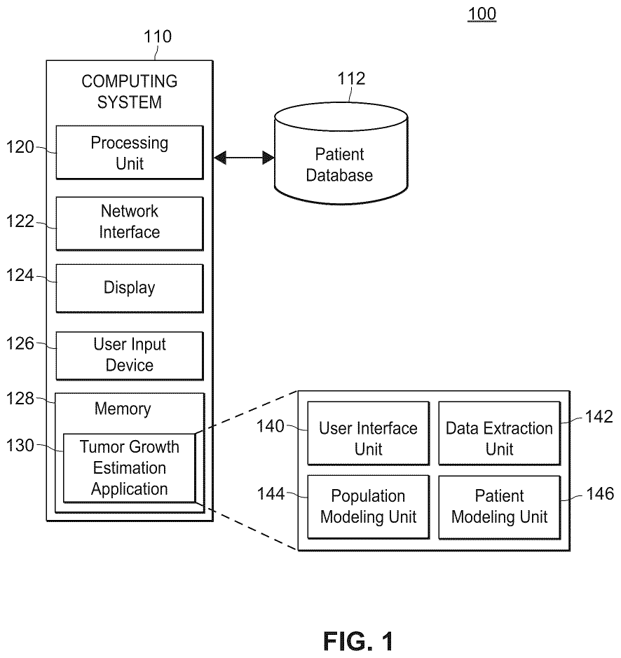

FIG. 1 is a simplified block diagram of an example system 100 that can implement the systems and methods (e.g., tumor growth modeling and estimation techniques) disclosed herein. System 100 includes a computing system 110 coupled to a patient database 112. Computing system 110 may be a single computing device, or include multiple co-located and/or distributed computing devices communicatively coupled by one or more networks. In the example embodiment shown in FIG. 1, computing system 110 includes a processing unit 120, a network interface 122, a display 124, a user input device 126, and a memory 128. Processing unit 120 includes one or more processors, each of which may be a programmable microprocessor that executes software instructions stored in memory 128 to execute some or all of the functions of computing system 110 as described herein. Alternatively, one, some or all of the processors in processing unit 120 may be other types of processors (e.g., application-specific integrated circuits (ASICs), field-programmable gate arrays (FPGAs), etc.), and the functionality of computing system 110 as described herein may instead be implemented, in part or in whole, in hardware. Memory 128 may include one or more physical memory devices or units containing volatile and/or non-volatile memory. Any suitable memory type or types may be used, such as read-only memory (ROM), solid-state drives (SSDs), hard disk drives (HDDs), and so on.

Network interface 122 may include any suitable hardware (e.g., front-end transmitter and receiver hardware), firmware, and/or software configured to communicate with external devices and/or systems (e.g., a client device, or one or more servers maintaining patient database 112) via one or more networks using one or more communication protocols. For example, network interface 122 may be or include an Ethernet interface, and/or include a wireless local area network (LAN) interface, etc.

Display 124 may use any suitable display technology (e.g., LED, OLED, LCD, etc.) to present information to a user, and user input device 126 may be a keyboard or other suitable input device. In some embodiments, display 124 and user input device 126 are integrated within a single device (e.g., a touchscreen display). Generally, display 124 and user input device 126 may combine to enable a user to interact with user interfaces (e.g., graphical user interfaces (GUIs)) provided by computing system 110, such as those discussed in further detail below. In some embodiments, however, computing system 110 does not include display 124 and/or user input device 126, or one or both of display 124 and user input device 126 are included in another computer or system that is communicatively coupled to computing system 110 (e.g., a client device not shown in FIG. 1).

Memory 128 stores the instructions of one or more software applications, including a tumor growth estimation application 130 (also referred to herein as “TGE application 130”). TGE application 130, when executed by processing unit 120, is generally configured to determine/learn distributions for one or more parameters (e.g., tumor growth model parameters), based on progression-free survival (PFS) data stored in patient database 112, and use the determined distribution(s) to determine/learn (jointly estimate) parameters of a pharmacokinetic-pharmacodynamic (PKPD) model for each of one or more specific patients. TGE application 130 can display the estimated tumor growth or any information associated with the estimated tumor growth. TGE application 130 includes a user interface unit 140, a data extraction unit 142, a population modeling unit 144, and a patient modeling unit 146. Generally, user interface unit 140 manages interactions with a user (e.g., a user operating user input device 126 and viewing display 124), data extraction unit 142 manages the retrieval of PFS data from patient database 112 (and/or any pre-processing of the PFS data), population modeling unit 144 learns distributions for baseline (i.e., no or ineffective drug treatment) tumor growth across a population of patients represented by the PFS data, and patient modeling unit 146 uses the learned population distributions, as well as tumor size observations over time for a particular patient, to model the contributions of both baseline tumor growth and drug treatment to the overall growth rate of the patient's tumor(s). The operation of TGE application 130 and its various units 140-146 is discussed in further detail below.

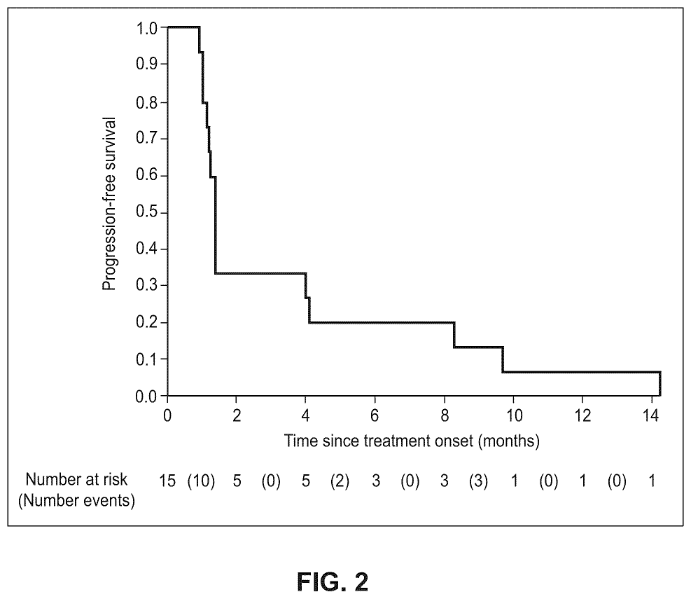

Patient database 112 may include one database or multiple databases, which may be stored in one or more memories at one or more co-located or remote locations. In some embodiments, patient database 112 is local to computing system 110 (e.g., stored in memory 128). Patient database 112 includes any information related to a plurality of patients, such as PFS data for a plurality of patients, for each of one or more cancer indications/types. For example, patient database 112 may include a digitized PFS plot and/or PFS risk table (e.g., of the sort represented in FIG. 2) for a particular cancer type, or may include multiple PFS plots and/or PFS risk tables each corresponding to a different cancer type (e.g., pancreatic, billary, breast, etc.). In another example, patient database 112 may include ORR.

Data extraction unit 142 is generally responsible for retrieving/obtaining the desired PFS data from patient database 112. In some embodiments, data extraction unit 142 retrieves PFS data for a particular type of cancer based on user input detected by user interface unit 140. For example, user interface unit 140 may generate and/or populate a GUI, and cause display 124 to present the GUI to a user. The user may then operate user input device 126 to enter an indication of a cancer type of interest via the GUI, and data extraction unit 142 may retrieve PFS data (e.g., a plot and/or risk table) corresponding to the indicated cancer type. In some embodiments, patient database 112 includes raw data (e.g., anonymized encounter data from health care providers indicating dates and diagnosis/measurements/etc.), and data extraction unit 142 constructs a PFS plot and/or PFS risk table, or some other similar data structure(s). For example, data extraction unit 142 may construct a digitized PFS plot from the raw data, or may generate data in a more readily usable form (e.g., an indexed list of patient-specific entries that each indicate the start and end of the time window in which the respective patient had a PFS event).

The PFS data obtained by data extraction unit 142 may be data that correspond to patients who have not had an effective drug treatment (e.g., patients treated with an experimental drug that was later shown to be ineffective, and/or patients who chose not to be treated with a drug at all). In this way, population modeling unit 144 can use the PFS data to learn “baseline” growth rate patterns for tumors of a particular cancer type.

Population modeling unit 144 uses the obtained PFS data (possibly after formatting, cleaning, and/or other pre-processing of the PFS data by data extraction unit 142) to determine a population distribution for one or more parameters (e.g., patient-specific parameters) of a growth curve function, i.e., to determine a distribution across the patient population that is represented by the obtained PFS data. To this end, population modeling unit 144 first uses the PFS data to learn the one or more parameters (e.g., one or more growth curve parameters) for each of the patients in the population. In some embodiments, a growth curve for a specific patient (patient i) as a function of time can be generically referred to herein as f(t, θᵢ), where θᵢ represents the one or more growth curve parameters specific to that patient (also referred to herein as “patient-specific parameter(s)” of the growth curve function). In some other embodiments, a growth curve for a specific patient (patient i) as a function of time can be generically referred to herein as R(t), where t represents time. In some embodiments, the growth curve function R(t) comprises one or more parameters (e.g., patient-specific parameters) comprise R₀⁽ⁱ⁾, k_{g}⁽ⁱ⁾, and f⁽ⁱ⁾, wherein t represents time and i is an index representing the i^{th} patient of the plurality of patients. In some embodiments, population modeling unit 144 fits a particular type of distribution (e.g., a log-normal distribution) to the censored observations of patients. Population modeling unit 144 learns the growth curve parameter(s) for a given patient as a distribution that allows for a range of possible growth curves. Notably, the information in a PFS plot or risk table can leave open the possibility that a PFS event occurred anywhere within a given time window. Referring to FIG. 2, for example, it can be seen from the risk table under the plot that two patients in the population had a PFS event somewhere between 4 and 6 months. That is, the PFS event for each of those two patients may have occurred at any time during the 2-month time window.

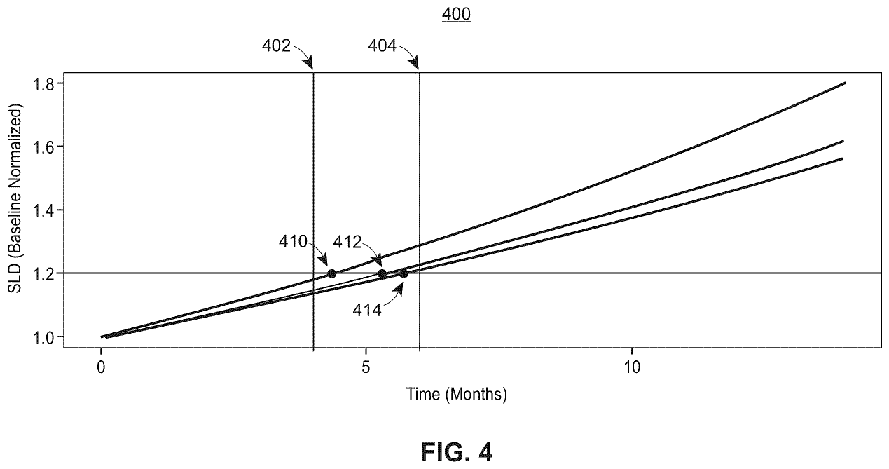

Some PFS plots can provide more granular information (e.g., as seen in FIG. 2, where the trace indicates the timing of PFS events with far more granularity than the 2-month windows of the PFS risk table). In such cases, population modeling unit 144 may be able to leverage more specific timing information to learn more accurate distributions of growth curve parameters. In some cases, however, the more granular timing is a result of factors that are not fully captured in the modeling (e.g., a patient becoming more ill and getting tested well before his/her next drug treatment), in which case population modeling unit 144 may instead ignore the more granular timing information of the PFS plot, and instead use the PFS risk table information. This can result in significant uncertainty with respect to the trajectory of the patient's growth curve. As seen in FIG. 4, for instance, a particular patient's PFS event, within the time window marked by start time 402 and end time 404, may have occurred at the time corresponding to PFS event 410, PFS event 412, or PFS event 414, or at any other time within the window. This uncertainty in each patient's estimate can affect the uncertainty in the estimate for the overall population. To avoid artificially removing that uncertainty (and thereby losing useful information), population modeling unit 144 does not assume a fixed time for each patient's PFS event, but instead learns the distribution(s) for the growth curve parameter(s) by directly using the starting and ending times of time windows in which the PFS events are known to have occurred (e.g., times 402 and 404 in FIG. 4) as censored observations. For example, from the PFS data (subject to measurement error) that the baseline-normalized SLD for a patient is less than 1.2 at time 402, and is at least 1.2 at time 404.

Population modeling unit 144 fits a particular type of distribution (e.g., a log-normal distribution) to the censored observations of patients. In one embodiment, for example, population modeling unit 144 assumes an exponential growth curve, such that:

f(t,θᵢ)=e^{k}^{_{g,i}}^{t}, (Equation 1)

where k_{g,i} is a patient-specific parameter controlling how quickly the growth curve accelerates, and t represents time. Population modeling unit 144 can then use the two censored observations to determine the population distribution for k_{g,i}. In another example embodiment, population modeling unit 144 assumes a logistic growth function with multiple parameters, e.g., such that:

$$f ( t , {θ}_{i} ) = \frac{{L}_{i}}{1 + {e}^{- {k}_{L , i} ( t - {t}_{0 , i} )}} , \qquad ( Equation 2 )$$

where Lᵢ is a patient-specific maximum value of the growth curve, k_{L,i} is a patient-specific parameter controlling the steepness of the growth curve, t is time, and t_{0,i} is the patient-specific time value at the midpoint of the growth curve. In this example, population modeling unit 144 can determine population distributions for the patient-specific parameters k_{L,i}, t_{0,i}, and Lᵢ (or possibly just k_{L,i} and t_{0,i}, with Lᵢ being constant across all patients, etc.). In still other embodiments, population modeling unit 144 can use any other suitable growth curve function and/or growth curve parameter(s).

Using y(t) to represent the baseline-normalized SLD, population modeling unit 144 may determine a population distribution of y(t) according to:

y(t)˜Log Normal(f(t,θᵢ),σ),

P(y(t)<1.2|f(t,θ),σ),

P(y(t)>1.2|f(t,θ),σ),

θᵢ˜Log Normal(μ,ω).

In the above expressions, a represents a noise parameter, which population modeling unit 144 may jointly estimate along with distribution(s) for the parameter(s) θᵢ. The parameters μ and ω are the mean and variance, respectively, of the learned log-normal distribution for θᵢ (e.g., with multiple means and variances if θᵢ includes more than one parameter). The probabilities (P) are probabilities/likelihoods that a PFS event (baseline-normalized SLD exceeding 1.2) has occurred for a given patient, or has not occurred for a given patient, at any time t. In particular, they are integrals of the probability density function of a log-normal distribution from 0 to 1.2 and from 1.2 to infinity, respectively.

In some embodiments, a growth curve for a specific patient (patient i) as a function of time can be generically referred to herein as R(t), where t represents time. In some embodiments, the growth curve function R(t) comprises one or more patient-specific parameters comprise R₀⁽ⁱ⁾, k_{g}⁽ⁱ⁾, and f⁽ⁱ⁾, wherein t represents time and i is an index representing the i^{th} patient of the plurality of patients. In some embodiments, population modeling unit 144 fits a particular type of distribution (e.g., a log-normal distribution) to the censored observations of patients. In one embodiment, for example, population modeling unit 144 assumes a growth curve as:

R(t)=R₀[fe^{k}^{ₛ}^{t}+(1−f)e^{k}^{_{g}}^{t}] (Equation 3)

where R₀ is the baseline SLD, k_{g} is the baseline growth rate in the absence of treatment, kₛ is the shrinkage/killing rates of drug sensitive tumor cells. In some embodiments, the killing rate of drug-sensitive tumors, kₛ, can be a fix constant that is an order magnitude faster than any plausible baseline growth rate k_{g}. This can align with empirical observations, although it can be further relaxed later. f is a proportion between zero and one representing what proportion of individual tumor cells are drug-sensitive.

To estimate the likelihood for a single subject, Xᵢₜ can comprise discrete SLD observation for the ith patient at the observation time t. The observations can be modeled as being log-normally distributed around the curve with some observational/model misspecification noise, σ:

Xᵢₜ|R₀⁽ⁱ⁾,k_{g}⁽ⁱ⁾,f⁽ⁱ⁾˜Log Normal(Log(R(tᵢ,R₀⁽ⁱ⁾,k_{g}⁽ⁱ⁾,f⁽ⁱ⁾)),σ)

where R₀⁽ⁱ⁾, k_{g}⁽ⁱ⁾, and f⁽ⁱ⁾ are the patient-specific parameters (e.g., individual-specific tumor growth/inhibition effects). In some embodiments, since Xᵢ₀ is the baseline, it can be modeled as having no noise and simply being R₀⁽ⁱ⁾.

The progression criteria can be the first time at which both of the following hold: 1. The SLD observation is 1.2 times greater than the minimum observed SLD; 2. The SLD observation is at least 5 mm larger than the minimum observed SLD.

At each observation point, the progression criteria can comprise a threshold that the observation is greater than to be considered progression. Formally this threshold can be max{1.2 min{Xᵢ₀, . . . ,X_{0,t-1}},min{Xᵢ₀, . . . ,X_{0,t-1}}+5}

If a patient is considered as having progressed at time Tᵢ then the likelihood of this patient's observations given their patient-specific parameters can be

p(Tᵢ|R₀⁽ⁱ⁾,k_{g}⁽ⁱ⁾,f⁽ⁱ⁾)=p(Xᵢ₁≤max{1.2,Xᵢ₀+5}|R₀⁽ⁱ⁾,k_{g}⁽ⁱ⁾,f⁽ⁱ⁾)

p(X_{iT}>max{1.2 min{Xᵢ₀, . . . ,X_{i,Ti-1}},min{Xᵢ₀, . . . ,X_{i,Ti-1}}+5}|R₀⁽ⁱ⁾,k_{g}⁽ⁱ⁾,f⁽ⁱ⁾)

If the patient is right-censored after time Ti−1, the last term can be removed since the patient's progression status is unknown at that time.

The above likelihood can involve a multi-dimensional integral with no known closed-form solution. The observations Xᵢₜ can be sampled while enforcing the constraints in Stan. Thus the likelihood can be the probability density function (PDF of each observation:

$$p ( {T}_{i} \vert {R}_{0}^{( i )} , {k}_{g}^{( i )} , {f}^{( i )} ) = {\prod }_{t = 1}^{T} p ( {X}_{i t} \vert {R}_{0}^{( i )} , {k}_{g}^{( i )} , {f}^{( i )} )$$

with the following constraints:

X_{iT}>max{1.2 min{Xᵢ₀, . . . ,X_{i,Ti-1}},min{Xᵢ₀, . . . ,X_{i,Ti-1}}+5}

for observations before the progression time, with the inequality being greater than for the observation at the progression time.

To generalize to population-level growth dynamics, the individual/patient-specific likelihood can be multiplied by the population distribution and the product over all patients can be:

$${\prod }_{t = 1}^{T} p ( {T}_{i} \vert {R}_{0}^{( i )} , {k}_{g}^{( i )} , {f}^{( i )} ) p ( {R}_{0}^{( i )} , {k}_{g}^{( i )} , {f}^{( i )} \vert T V {R}_{0}^{( i )} , {ω}_{R 0} , {TVk}_{g}^{( i )} , {ω}_{k g} , TV {k}_{g}^{( i )} , {TVf}^{( i )} )$$

Rather than marginalize out patient-specific parameters, patient-specific parameters can be sampled in Stan in addition to the patient-specific observation and the population observations.

ORR information from the historical study can be included into the likelihood. The ORR, or equivalently, the number of patients who experience a response can be given. The overall response rate can be the smallest observation for a patient's tumor being smaller than 0.7 times the baseline. If the minimum observation for every patient is, Mi:=min{Xᵢ₁, . . . ,X_{iT}}, then the ORR can be that

$$\frac{1}{N} {\sum }_{k = 0}^{n} I ( {M}_{i} < 0.7 ) = ORR$$

In some embodiments, an approach using soft constraints that are amenable can be used to sample in Stan and Approximate Bayesian Inference can be:

\tilde{I}(Mᵢ<0.7)=1−log it⁻¹(κ(Mᵢ−0.7))

where κ controls the smoothness of this approximation and hand-tune in a range of 10 to 50 (e.g., 20). In some embodiments, the likelihood with the density can be

$$\frac{1}{N} {\sum }_{k = 0}^{n} \tilde{I} ( {M}_{i} < 0 \text{.7} ) ∼ N ( ORR , τ )$$

where τ is tuned to enforce the ORR of each posterior sample of the entire dataset to be close to the true observed ORR. Since the ORR is a discrete number this yields posterior sampled that mostly satisfy the ORR constraint, but a few that may not. The method disclosed herein can use posterior sampling, and enforce the ORR condition with a soft constraint. In some embodiments, all posterior samples can be rejected where the ORR constraint is not satisfied Approximate Bayesian Computation (ABC).

In some embodiments, progression can comprise a situation as when SLD reaches a certain threshold. In some other embodiments, progression criteria can comprise more than a situation as when SLD reaches a certain threshold, but can also be due to a nontarget progression event such as appearance of a new lesion, the progression of a nontarget lesion, symptomatic deterioration, or death. To account for this, these nontarget progression events can be modeled using a hazard function that is itself a function of tumor growth inhibition dynamics using the form as below:

h(t|R₀⁽ⁱ⁾,k_{g}⁽ⁱ⁾,f⁽ⁱ⁾)=exp{λ₀+λ₁R(t|R₀⁽ⁱ⁾,k_{g}⁽ⁱ⁾,f⁽ⁱ⁾)+λ₂R′(t|R₀⁽ⁱ⁾,k_{g}⁽ⁱ⁾,f⁽ⁱ⁾)}

The hazard function can comprise a survival function S(t|R₀⁽ⁱ⁾,k_{g}⁽ⁱ⁾,f⁽ⁱ⁾) that can be incorporated into the individual patient likelihood. Specifically, for a patient with progression at time T, their SLD observation at that time, X_{iT}, may not be greater than the SLD progression threshold and is thus no longer lower bounded. However, if X_{iT} is below the threshold and the patient have progression at time T, then the patient may have nontarget progression events during the interval between T−1 and T. Thus likelihood can be augmented by

S(T−1|R₀⁽ⁱ⁾,k_{g}⁽ⁱ⁾,f⁽ⁱ⁾)−S(T|R₀⁽ⁱ⁾,k_{g}⁽ⁱ⁾,f⁽ⁱ⁾)

Otherwise, if X_{iT} is greater than the SLD progression threshold, then the patient may not have had nontarget progression before time T, but the possibility is not precluded and thus their likelihood is augmented by the term

S(T−1|R₀⁽ⁱ⁾,k_{g}⁽ⁱ⁾,f⁽ⁱ⁾)

In some embodiments, a GUI provided by user interface unit 140 enables users to select or enter a desired type of growth curve function and/or growth curve parameter(s) (e.g., from a drop-down list of options), and population modeling unit 144 uses the selected function/parameter(s). In still other embodiments, the GUI provided by user interface unit 140 enables users to select a specific cancer type, after which data extraction unit 142 obtains (from patient database 112) PFS data corresponding to a patient population with that cancer type, and population modeling unit 144 automatically selects a growth function that is a priori to be suited for the selected cancer type.

As with the growth function, population modeling unit 144 may assume any suitable type of distribution for the distribution across the patient population represented by the PFS. For example, for the θᵢ log-normal distribution above, the indication-specific parameter μ may itself have follow a distribution μⱼ˜Log Normal(α,τ), or the distribution μⱼ˜Log Normal(α+βxᵢ,τ) where xᵢ is an indication or study-specific covariate, and so on. In some embodiments, the GUI provided by user interface unit 140 enables users to select or enter a desired type of distribution for the parameter(s) θᵢ (e.g., from a drop-down list of options), and population modeling unit 144 uses the selected distribution.

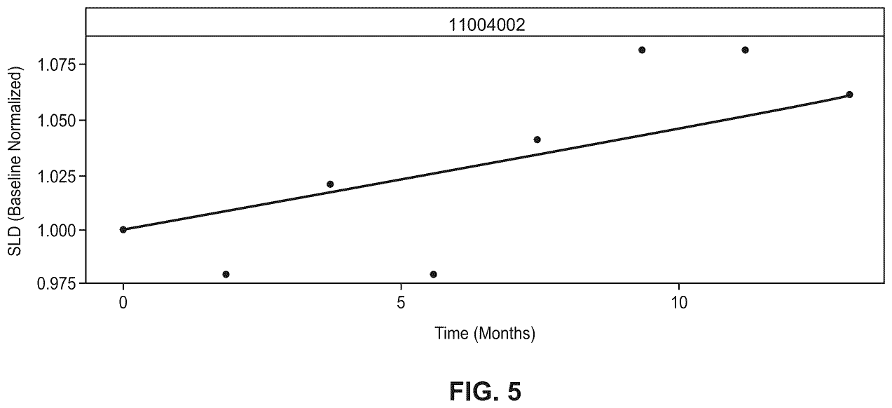

Directly modeling from censored observations (i.e., two censored observations per patient in the population), as described above, can provide several advantages. With the direct modeling approach, the uncertainty inherent to PFS plots and risk tables (discussed above) can be more accurately quantified, leading to a more accurate picture of when PFS events are likely to occur. The system and methods can better account for other sources of uncertainty, such as SLD measurement/observation noise. Noisy SLD measurements can be very common, and can result, for example, in a PFS event being detected in the wrong time window (e.g., if the PFS event actually occurs just before the time window began, but the measurements for the tumor size(s) is/are slightly off). An example of SLD noise is shown in FIG. 5, with each dot representing a different observation/measurement.

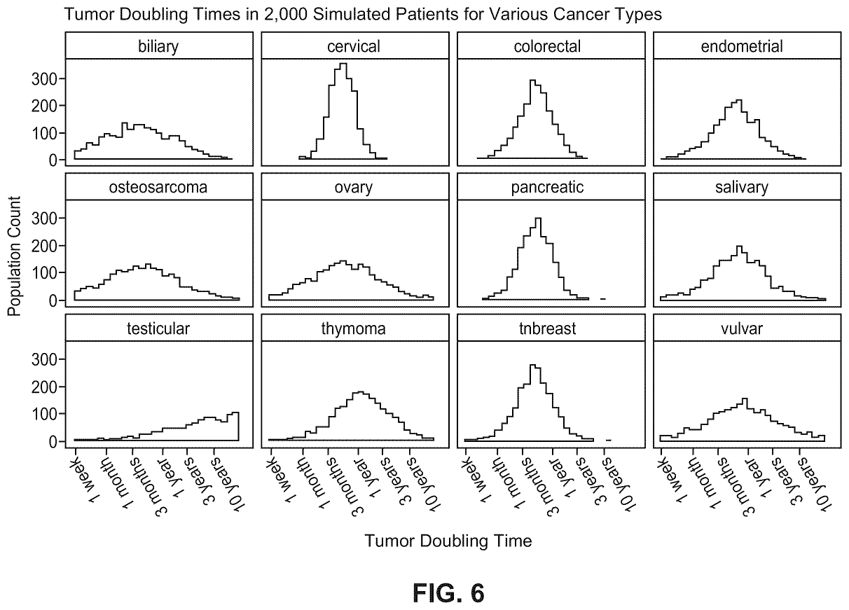

Moreover, the direct modeling approach is highly flexible, allowing for different growth curve functions and/or distribution types, as discussed above. By directly modeling from the PFS data in a probabilistic programming language, for example, the growth curve can take any form so long as the trajectory to plug into the likelihood/probability can be forward-solved. Different studies can also be modeled as having a non-zero effect, so long as certain assumptions are made about particular PKPD characteristics. Population distributions of growth curves can likewise be generalized in a probabilistic programming language. More flexible distributions, with more parameters or particular characteristics (e.g., biomodal, sparse, or heavier-tailed) can be used, for example. Indication-level parameters may come from a hierarchical distribution across indications (cancer types), allowing for pooling of information (especially for less-informed studies). Population modeling unit 144 may regress indication-level parameters, e.g., to model progression rates as a function of previous lines of treatments. Some example population distributions for different cancer/disease indications/types are shown in FIG. 6.

As yet another advantage, joint estimation of parameters (e.g., one or more parameters θᵢ as well as a noise parameter σ) can better inform the learning of those parameters.

After population modeling unit 144 determines/learns the parameter distribution(s) for baseline tumor growth across the population represented by the PFS data, patient modeling unit 146 uses the distribution(s), as well as SLD observations/measurements over time for a particular patient, to model the contributions of both baseline tumor growth and drug treatment to the overall tumor growth rate for that patient. More specifically, patient modeling unit 146 jointly estimates, using a PKPD model, parameters of expressions that comprise characteristics of both these contributions. The patient for which the PKPD model is applied can be a patient having the same type of cancer as the patients reflected in the PFS data, or possibly a type of cancer known to have very similar tumor growth characteristics.

As noted above, the techniques described herein may assume or apply any suitable type of growth curve (exponential, logistic, etc.). In some embodiments where exponential growth is assumed, patient modeling unit 146 may jointly estimate tumor growth parameters for a particular patient using the following PKPD model:

$${T}^{′} = {k}_{g} T - {k}_{max} T \frac{C}{E C 50 + C} , \qquad ( Equation 4 )$$

where T′ is the tumor growth rate for the patient, T is the tumor size for the patient, k_{g} is the exponential growth parameter for the patient (e.g., per Equation 1), kₘₐₓ, is the max kill rate for the patient, C is the drug concentration in plasma for the patient, and EC50 is the half-maximal effective concentration for the patient. In some embodiments, the patient modeling unit 146 models tumor growth for the patient using an equation with more or fewer parameters and/or having a different format. For example, each instance of C in Equation 4 may be multiplied by another tumor perfusion parameter p, representing a proportion of the concentration of the drug at the patient's tumor site(s) to the concentration of the drug in the patient's plasma. The perfusion parameter may also be jointly estimated, or a fixed value may be assumed (e.g., depending on cancer type), etc.

Patient modeling unit 146 may learn the patient-specific parameters of Equation 4 (or patient-specific distributions for the parameters of Equation 4) by attempting to fit Equation 4 to measured/observed tumor sizes/growth for the patient (i.e., over time and during a series of two or more patient encounters), subject to certain constraints. For example, if exponential growth is assumed, patient modeling unit 146 may use the determined population distribution of k_{g} to set constraints for k_{g} in Equation 4. As a more specific example, patient modeling unit 146 may determine the upper and lower values of k_{g} that correspond to a 90% certainty interval in the population distribution, and then use those values as upper and lower bounds when estimating k_{g} for the particular patient.

Patient modeling unit 146 can also use informative priors to set constraints on one or more other parameters in Equation 4. For example, kₘₐₓ may have an informative prior of 0.025 (e.g., for the mean of a log-normal distribution), as informed by the fastest responding patient in a population, but is also allowed to vary from this value in the joint estimation process.

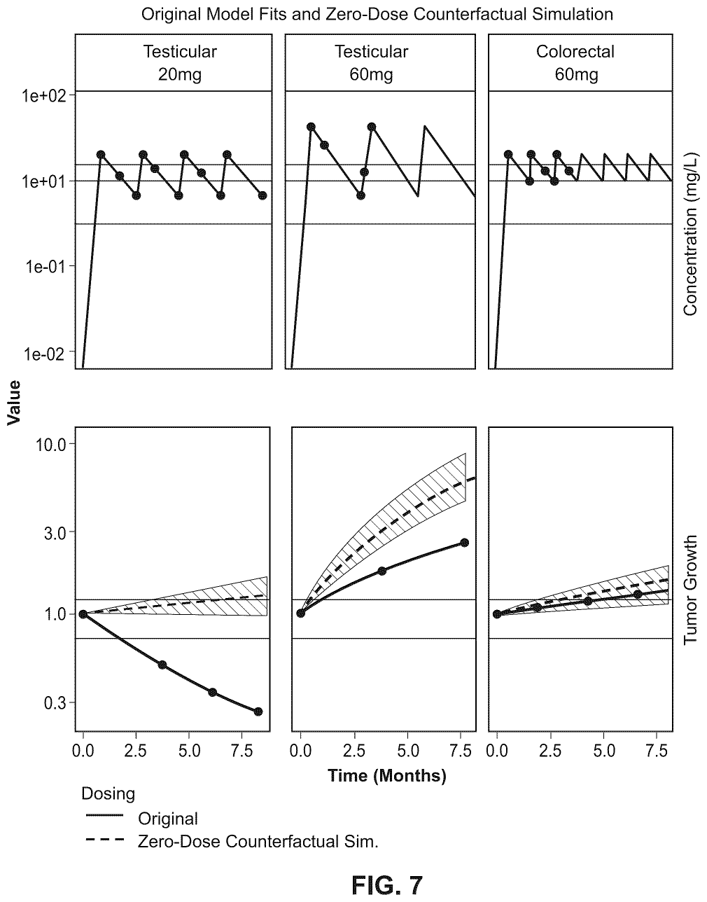

Once patient modeling unit 146 has estimated the parameters and/or parameter distributions, user interface unit 140 can cause a display (e.g., display 124) to present a visual indication of the estimated tumor growth for the particular patient, in any suitable format. For example, for a particular patient, user interface unit 140 may output the plots/information shown in any one of the columns shown in FIG. 7. In FIG. 7, each column represents, for a particular patient, a particular combination of drug dose (in this example, 20 mg or 60 mg) and cancer indication (in this example, testicular or colorectal).

In the top row of each column, each dot represents an observed/measured concentration of the drug in the respective patient's plasma, while the solid line/trace represents the mean value of concentration in plasma that is estimated by patient modeling unit 146 using a PKPD model similar to Equation 4. While not shown in FIG. 7, shaded zones around the solid lines/traces may be displayed (e.g., to represent a 90% confidence interval for concentration in plasma). In the bottom row of each column, each dot represents an observed/measured, baseline-normalized SLD, and the top horizontal line indicates a baseline-normalized SLD value of 1.2. The solid lines/traces in the bottom row represent the mean value of the estimated, baseline-normalized SLD for the respective patient, and the dashed lines/traces in the bottom row represent the estimated, baseline-normalized SLD for the respective patient in a counterfactual scenario where the drug was not administered to the patient (i.e., the estimated tumor growth for the patient in the absence of the drug treatment). In the bottom row, the solid lines/traces correspond to the T′ of Equation 4, while the dashed lines/traces correspond to the first term on the right side of Equation 4. The shaded zones around the dashed lines represent 90% confidence intervals. While not shown in FIG. 7, the solid lines may also have surrounding shaded zones to represent 90% confidence intervals.

By showing a trajectory of tumor growth for the particular patient in the counterfactual scenario where the particular patient had not received the drug treatment, relative to the actual trajectory (i.e., relative to real-world observations and/or estimated total growth rate), plots such as those shown in FIG. 7 can enable users to accurately assess the efficacy of the drug at the indicated dosing level and against the indicated cancer type. For example, a user may be able to determine that for 60 mg/colorectal, the low overall growth rate is due to the low baseline tumor growth in that patient, much more so than the efficacy of the drug/dose in that patient. On the other hand, for 20 mg/testicular, a user can determine that the negative growth rate is largely due to drug efficacy, given the positive baseline growth that otherwise would have occurred. And for 60 mg/testicular, a user can determine that the drug is efficacious despite the rather high growth rate, because the baseline growth rate is significantly higher than that growth rate.

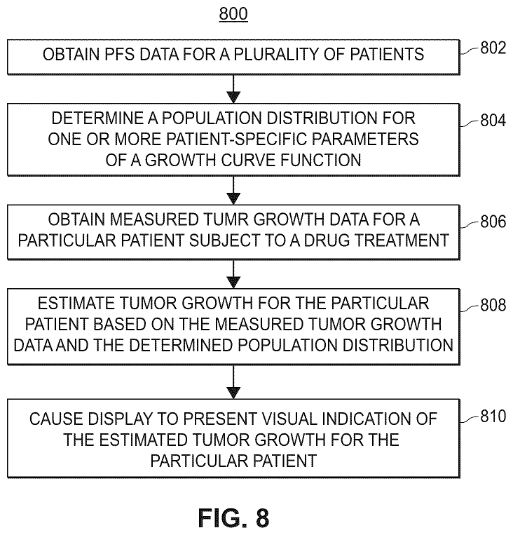

FIG. 8 is a flow diagram of an example method 800 for estimating tumor growth. Method 800 may be performed by computing system 100 (e.g., processing unit 120) when executing instructions of TGE application 130, for example. In some embodiments, the method 800 can comprise a Bayesian generative model.

The method 800 includes step 802, in which one or more processors (e.g., one or more processors of the computing system 110) may obtain progression-free survival (PFS) data for a plurality of patients (e.g., from patient database 112 or a local memory). The patient (or subject) can comprise any living or non-living organism, including but not limited to a human (e.g., a male human, female human, fetus, pregnant female, child, or the like), a non-human animal, a plant, a bacterium, a fungus or a protist. Any human or non-human animal can serve as a patient, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a patient (or subject) is a male or female of any stage (e.g., a man, a women or a child). The plurality of patients may be patients that are associated with one or more drug treatments. The one or more drug treatments may include effective drug treatment (e.g., successful trials), neutral drug treatment, or ineffective drug treatments (e.g., failed trials). Details of the one or more drug treatments are described elsewhere herein.

The Progression-free survival (PFS) can comprise the time from initiation of treatment to the occurrence of disease progression or death. In some embodiments, disease progression can be evaluated by the Response Evaluation Criteria in Solid Tumors (RECST) as an increase in the sum of maximum tumor diameters of at least 20%, the development of any new lesions, or an unequivocal increase in non-measurable malignant disease. In some embodiments, the PFS can be used as an endpoint. An endpoint can comprise a targeted outcome of a clinical trial to determine the efficacy and safety of the therapy being studied. An endpoint for a clinical trial may include one or more clinical outcome assessment and/or surrogate endpoint. In some embodiments, the clinical outcome assessment can comprise cure, clinical worsening, and mortality. The surrogate endpoint can be a clinical trial endpoint used as a substitute for a direct measure of how a patient feels, functions, or survives. The PFS data can comprise any information associated with PFS, including but not limited to a PFS plot, a digitized PFS plot, or a PFS risk table, as described elsewhere herein. The PFS data can comprise published PFS data or unpublished PFS data. The PFS data can indicate how many of the plurality of patients, at each of the plurality of observation times and/or within the most recent time window, have a baseline-normalized sum-of-longest diameters (SLD) measurement of at least 1.2 or have new lesions appear. The plurality of patients represented by the PFS data can comprise patients known to be associated with one or more drug treatments, as described elsewhere herein.

The PFS data can indicate (i) a plurality of observation times, and/or (ii) how many of the plurality of patients, at each of the plurality of observation times, had a PFS event. The plurality of observation times can comprise any time range, for example, at least one month, two months, three months, four months, five months, six months, one year, five years or longer. In some embodiments, the plurality of observation times can comprise any time range, for example, at most five years, one year, six months, five months, four months, three months, two months, one month or shorter. The plurality of observation times can be at least 1 hour, 1 day, 1 month, 1 quarter, 1 year or longer. In some other embodiments, the plurality of observation times can be at most 1 year, 1 quarter, 1 month, 1 day or shorter. The PFS event can comprise any even associated with PFS, including, but not limited to, the progression of non-target lesions, a SLD observation that a baseline-normalized SLD is 1.2 times greater than the minimum observed SLD, a SLD observation that a baseline-normalized SLD is at least 5 mm larger than the minimum observed SLD, or a situation having new lesions appear within a most recent time window. The most recent time window can be the same as or different from the plurality of observation times. The most recent time window can be at least 1 hour, 1 day, 1 month, 1 quarter, 1 year or longer. In some other embodiments, the most recent time window can be at most 1 year, 1 quarter, 1 month, 1 day or shorter. The number of the plurality of patient who at each of the plurality of observation times had a PFS event can be at least 10, 100, 1000, 10000 or more. The number of the plurality of patient who at each of the plurality of observation times had a PFS event can be at most 10000, 1000, 100, 10 or less. In some embodiments, the PFS data can comprise how many of the plurality of patients, at a subset of the plurality of observation times, had a PFS event. The subset of the plurality of observation times can be at least 10%, 20%, 30%, 40%, 50%, 60%, 70%, 80%, 90% or more of the plurality of observation times. In some embodiments, the subset of the plurality of observation times can be at most 90%, 80%, 70%, 60%, 50%, 40%, 30%, 20%, 10% or less of the plurality of observation times. The PFS data may correspond to one or more particular cancer types, The one or more particular cancer types can comprise but are not limited to breast cancer, colorectal cancer, esophageal cancer, head/neck cancer, lung cancer, a lymphoma, ovarian cancer, pancreatic cancer, prostate cancer, renal cancer, or uterine cancer.

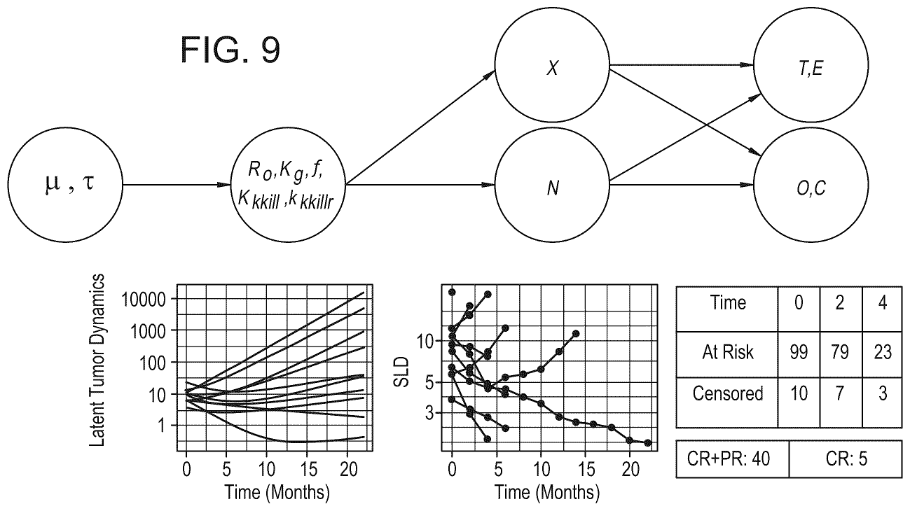

In step 804, the one or more processors can determine a population distribution for one or more patient-specific parameters of a growth curve function. The population distribution can comprise a distribution of one or more parameters or characteristics (e.g., patient-specific parameters) among a plurality of individuals in a population. The one or more parameters can comprise patient-specific parameters and non-patient-specific parameters (e.g., population parameters). The one or more patient-specific parameters can comprise tumor growth parameters, PKPD parameters, or growth curve parameters. The one or more patient-specific parameters can comprise individual parameters, SLD observations, or nontarget progression events. Population parameters can be determined by the population e.g. chemo-naïve prostate cancer patients in a phase 3 study of antiandrogen therapies. For example, in FIG. 9, the population parameters can comprise μ˜HalfNormal(0,0.1) and τ˜HalfNormal(0,1). The individual/patient-specific parameters can be drawn conditional on the population parameters. For example, in FIG. 9, the individual/patient-specific parameters can comprise R₀˜Log Normal(μ_{R}_{₀},τ_{R}_{₀}), k_{g}˜Reg Log Normal(μₖ_{_{g}},τₖ_{_{g}},c) and f˜Log itNormal(μ_{f},τ_{f}). The SLD observations and nontarget progression event times can be drawn conditional on the dynamics based on the individual/patient-specific parameters. For instance, in FIG. 9, SLD observation can be Xₜ˜Log Normal(R(t),σ) and nontarget progression event can be N˜h(t)=exp{λ₀+λ₁R(t)+λ₂R′(t)}.

In some embodiments, determining the population distribution for the one or more patient-specific parameters can comprise determining a growth curve function comprising the one or more patient-specific parameters. The growth curve function can comprise an exponential growth function, a logistic growth function, or an ordinary differential function. The growth curve function can comprise Equation 1, Equation 2, or Equation 3 as disclosed elsewhere herein. Details of the growth curve function are described elsewhere herein. Population distributions of growth curves can be generalized in a probabilistic programming language. The growth curve function can be time-dependent. In this situation, the growth curve function can vary based on the plurality of observation times. In some embodiments, the method can further comprise determining, via the one or more processors, a population distribution for one or more parameters.

In some embodiments, the one or more patient-specific parameters can comprise θᵢ and a growth curve function associated with θᵢ can be f(t, θᵢ), as described elsewhere herein. In some embodiments, θᵢ of a growth curve function f(t, θᵢ) can be determined, where t represents time, and i is an index representing the i^{th} patient of the plurality of patients. If exponential growth is assumed, for example, the patient-specific parameter(s) θᵢ may include the parameter k_{g}, and the growth curve function may be as shown in Equation 1. In some embodiments, the one or more patient-specific parameters can comprise R₀⁽ⁱ⁾, k_{g}⁽ⁱ⁾, and f⁽ⁱ⁾ and a growth curve function associated with R₀⁽ⁱ⁾, k_{g}⁽ⁱ⁾, and f⁽ⁱ⁾ can be R(t), as described elsewhere herein. In some embodiments, R₀⁽ⁱ⁾, k_{g}⁽ⁱ⁾, and f⁽ⁱ⁾ of the growth function R(t) can be determined, wherein t represents time and i is an index representing the i^{th} patient of the plurality of patients. In some embodiments, R₀⁽ⁱ⁾ can comprise a baseline-normalized sum-of-longest diameters (SLD) measurement. In some embodiments, the f⁽ⁱ⁾ can comprise a proportion of drug-sensitive tumor cells in the i^{th} patient of the plurality of patients. In some embodiments, the f⁽ⁱ⁾ can range from 0 to 1. In some embodiments, the growth curve function is a logistic growth function; and the one or more patient-specific parameters include a plurality of parameters of the logistic growth function.

In various embodiments, determining the population distribution for the one or more patient-specific parameters can comprise determining a single population distribution for a single patient-specific parameter, determining respective population distributions for two or more patient-specific parameters, or determining a single, joint population distribution for two or more patient-specific parameters. The one or more patient-specific parameters can comprise a parameter for baseline-normalized sum-of-longest diameters (SLD) measurement. The baseline-normalized SLD measurement can be determined based on PSF data. Details of the baseline-normalized SLD measurement are described elsewhere herein. The one or more patient-specific parameters can comprise a parameter for growth rate (e.g., tumor growth parameter). The parameter for growth rate can comprise a parameter for baseline growth rate with or without treatment. The one or more patient-specific parameters can comprise a parameter for a proportion of drug-sensitive tumor cells in a patient of the plurality of patients. A value of the parameter for a proportion of drug-sensitive tumor cells in the patient of the plurality of patients ranges from 0 to 1. In some embodiments, the value of the parameter for a proportion of drug-sensitive tumor cells in the patient of the plurality of patients can be at least 10%, 20%, 30%, 40%, 50%, 60%, 70%, 80%, 90% or more. In some embodiments, the value of the parameter for a proportion of drug-sensitive tumor cells in the patient of the plurality of patients can be at most 90%, 80%, 70%, 60%, 50%, 40%, 30%, 20%, 10% or less.

Determining the population distribution for the one or more patient-specific parameters can include fitting the growth curve function to observations at the plurality of observation times. Determining the one or more population distributions includes fitting the growth curve function to observations at a subset of the plurality of observation times. The subset of the plurality of observations times can be selected automatically via a mathematical model or manually by one or more users. For example, the observations may include, for each patient of the plurality of patients, a first observation at a first time indicating that the patient did not have a PFS event before the first time, and a second observation at a second time indicating that the patient did have a PFS event before the second time. The population distribution(s) may include any type of distributions, including but not limited to, a log-normal distribution, a Bernoulli distribution, a uniform distribution, a binomial distribution, a normal or Gaussian distribution, an exponential distribution, or a Poisson distribution.

In step 806, one or more processors can obtain measured tumor growth data for a particular patient subject to a drug treatment (e.g., from patient database 112 or a local memory such as memory 128). The tumor growth data can comprise any information related to growth of the tumor, including, but not limited to, the diameter of the tumor over time, the volume of the tumor over time, or the rate of tumor growth. The tumor growth data can be obtained by any type of technique such as imaging studies including mammography, ultrasound (US), and magnetic resonance imaging (MRI). The drug treatment can comprise one or more cancer therapeutic agents, including but not limited to, a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents comprising monoclonal antibody therapies.

In step 808, one or more processors can estimate tumor growth for the particular patient (e.g., a patient having the same cancer type as the plurality of patients) based on (i) the measured tumor growth data and (ii) the determined population distribution. Step 808 may include modeling a tumor growth rate for the particular patient using a PKPD model (e.g., Equation 4 or similar) having a first term representing tumor size variation in the particular patient without the drug treatment and a second term representing a contribution to tumor size variation in the particular patient due to the drug treatment, and jointly estimating one or more parameters of the first term and one or more parameters of the second term by fitting the PKPD model to the measured tumor growth data. The joint estimation may include using the population distribution(s) determined at block 804 to set constraints for the one or more parameters of the first term (e.g., k_{g}). The parameter(s) of the second term may include drug concentration in plasma of the patient, kₘₐₓ (a max kill rate) for the patient, and/or half-maximal effective concentration (EC50), for example. The constraints can comprise any population priors.

Estimating the tumor growth can further comprise obtaining an overall response rate (ORR) for a particular patient subject to a drug treatment. The ORR can comprise a number of patients who experience a response. Response can comprise the observation that a patient's tumor growth satisfies one or more response conditions. The response conditions can comprise the patient's tumor size being smaller than 0.7 times the baseline SLD (or 30% smaller than their baseline SLD). In some embodiments, the ORR can be calculated for each sample and fixed to a value. For instance, exact inference can sum over all combinatorial possibilities of responders. Instead, this can be approximated by setting the simulated proportion of responders close the observed:

$${\overline{O}}_{o b s} ∼ N ( \frac{1}{N} Σ {O}_{i} , {σ}_{O} ) .$$

The simulated responder status of each patient can be represented by an inverse logit that approximates the response condition: whether or not a patient had an SLD measurement, Xₜ, 30% smaller than their baseline SLD (response condition). In some embodiments, estimating the tumor growth can further comprise obtaining one or more non-patient specific parameters. In this situation, the non-patient-specific parameters can comprise fixed values. Such non-patient-specific parameters can comprise killing rate.

Estimating the tumor growth further can comprise obtaining one or more nontarget progression events for a particular patient subject to a drug treatment. In situation where progression is not due to SLD reaching a certain threshold, a nontarget progression event, such as appearance of a new lesion, the progression of a nontarget lesion, symptomatic deterioration, or death, can be included into population distribution or growth curve to determine the tumor growth. The one or more non-target progression events can be included by modeling the time to these nontarget events using a hazard function. The hazard function can comprise a function of tumor growth inhibition dynamics. In some embodiments, the PFS data corresponds to a specific cancer type; and estimating tumor growth for the particular patient can include estimating tumor growth for a patient diagnosed with the specific cancer type.

In step 812, one or more processors can cause a display (e.g., display 124) to present a visual indication of the estimated tumor growth for the particular patient. Step 812 may include generating and/or populating a user interface, for example. In some embodiments, step 812 includes causing the display to show a trajectory of tumor growth for the particular patient if the particular patient had not had the drug treatment. In this situation, the visual indication of the estimated tumor can be presented on a user interface to the health/research professionals, and health/research professionals can prescribe one or more drug treatments. In some other embodiments, step 812 includes causing the display to show a trajectory of tumor growth for the particular patient if the particular patient had one or more drug treatments. In this situation, the visual indication of the estimated tumor can be presented on a user interface to the health/research professionals, and health/research professionals can adjust dose, regimen, or anti-drug antibodies associated with the one or more drug treatments. In this situation, such change of dose, regimen, or anti-drug antibodies associated with one or more drug treatments can be displayed, via one or more processors, to the patient.

The methods and systems disclosed herein may further comprise adjusting a dose of the drug treatment based on the estimated tumor growth for the particular patient. For instance, if the tumor growth for the particular patient is lower than a predetermined threshold (e.g., a predetermined tumor growth rate), then the dose of the drug treatment may be adjusted to a lower dose. In another example, if the tumor growth for the particular patient is higher than a predetermined threshold, then the dose of the drug treatment may be adjusted to a higher dose. Additionally, in some embodiments, the estimated tumor growth for the particular patient can be used to assess an efficacy of a drug treatment, assess an efficacy of a combination of one or more drug treatments, or compare the head-to-head results of each drug treatment as a monotherapy and as a combination of one or more drug treatments.

Additional considerations pertaining to this disclosure will now be addressed.

Some of the figures described herein illustrate example block diagrams having one or more functional components. It will be understood that such block diagrams are for illustrative purposes and the devices described and shown may have additional, fewer, or alternate components than those illustrated. Additionally, in various embodiments, the components (as well as the functionality provided by the respective components) may be associated with or otherwise integrated as part of any suitable components.

Embodiments of the disclosure relate to a non-transitory computer-readable storage medium having computer code thereon for performing various computer-implemented operations. The term “computer-readable storage medium” is used herein to include any medium that is capable of storing or encoding a sequence of instructions or computer codes for performing the operations, methodologies, and techniques described herein. The media and computer code may be those specially designed and constructed for the purposes of the embodiments of the disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as optical disks; and hardware devices that are specially configured to store and execute program code, such as ASICs, programmable logic devices (“PLDs”), and ROM and RAM devices.

Examples of computer code include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel. Another embodiment of the disclosure may be implemented in hardwired circuitry in place of, or in combination with, machine-executable software instructions.

### EXAMPLES

### Bayesian Generative Model of Published PFS and ORR Data

Methods and systems disclosed herein were developed for estimating tumor growth. In some embodiments, a Bayesian Generative model was developed specifying the joint distribution of one or more parameters including trial-specific parameters, arm-specific parameters, individual patient parameters, individual SLD observations, non-target progression events, and reported PFS and ORR in a published study. The model included a semi-mechanistic population component to capture longitudinal tumor dynamics at the individual and population level. Furthermore, the model included a novel component based on RECIST criteria that specified an individual's distribution of progression time and response conditional on their individual tumor dynamics. The RECIST criteria was the criteria to determine whether a tumor disappears, shrinks, stays the same or gets bigger, including, complete response (CR), partial response (PR), stable disease (SD) and progressive disease (PD). The joint distribution allowed for joint estimation of one or more parameters conditioned on observed data from published studies using standard Bayesian estimation.

Individual tumor dynamics were described using a standard two-state model representing two tumor subpopulations each with linear growth and killing:

R′₁=k_{g}R₁−kₖₖᵢₗₗR₁

R′₂=k_{g}R₂−kₖₖᵢₗₗR₂.

Here R₁ was a parameter representing a subpopulation of drug-sensitive cells and R₂ represented a subpopulation of more resistant cells (e.g., drug-insensitive cells) with killing rate kₖₖᵢₗₗᵣ<kₖₖᵢₗₗ (kₖₖᵢₗₗᵣ is the killing rate for more resistant cells and kₖₖᵢₗₗ is the killing rate for drug-sensitive cells). The baseline growth rate was k_{g}. The subpopulation was associated with a proportion of drug-sensitive tumor cells in a patient of the plurality of patients. Overall tumor burden/function, R(t), was equal to the weighted sum of R₁(t) and R₂(t) with a parameter f determining the proportion of drug-sensitive tumor cells in a patient of the plurality of patients and the proportion of drug-insensitive tumor cells in the patient of the plurality of patients. The linear model thus admitted the following closed form solution in the familiar biexponential form:

R(t)=R₀[fe^{k}^{ₛ}^{t}+(1−f)e^{k}^{ᵣ}^{t}]

where R₀ was the initial SLD, kₛ:=k_{g}−kₖₖᵢₗₗ, and kᵣ:=k_{g}−kₖₖᵢₗₗᵣ.

Initial SLD, growth rates, and drug-sensitive cell proportions were individual-specific parameters (or patient-specific parameters) and were distributed according to the following distributions for patients in the same study regardless of arm:

R₀˜Log Normal(μ_{R}_{₀},τ_{R}_{₀})

k_{g}˜Reg Log Normal(μₖ_{_{g}},τₖ_{_{g}},c)

f˜Log itNormal(μ_{f},τ_{f})

Where σ can be, and τₖ_{_{g}} Reg Log Normal(μ, σ, c) denoted a regularized log-normal distribution so that if

$$Y ∼ N ( μ , τ ) then X := \frac{c {e}^{y}}{c + {e}^{y}} ∼ RegLogNormal ( μ , σ , c ) .$$

This distribution behaved as a log-normal distribution on (0, c). For numerical stability and to avoid unrealistically large values of k_{g}, c was fixed to 0.02/day which corresponded to a progression time of log(1.2)/0.02≈9 days. kₖₖᵢₗₗ and kₖₖᵢₗₗᵣ, were not modelled as patient-specific and were determined by the arm of a study that a patient belongs to. In practice, a fixed value of the killing rate parameters within an arm was adequate to describe the data and was reasonable in later-stage trials where doses within an arm were standardized.

SLD observations, Xₜ, at time t were modelled as lognormally distributed about R(t) conditional on the patient-specific parameters:

Xₜ˜Log Normal(R(t),σ).

The time-to-event of non-target progression events, N, which was the time to the progression of a non-target lesion or appearance of a new lesion as per RECIST criteria, was modeled conditional on patient-specific parameters via a hazard model based on the following:

h(t)=exp{λ₀+λ₁R(t)+λ₂R′(t)}.

The hazard was modeled as proportional to both the absolute tumor burden or growth rate and its rate of change. This captured the situation that the dynamics of non-target lesions were correlated to target lesions and that the appearance of new lesions was intuitively more likely when existing tumor burden was large and growing. While the parameters σ and λ were not identifiable by PFS and ORR data alone, their values, or at least prior distributions on their values, were obtained by fitting the model on actual SLD data which was often not public available. The parameters were set to the following fixed values obtained from point estimates of a model fitting on private SLD data from an internal program: σ=0.1, λ₁=−6.4, λ₂=0.014, λ₂=2.6. For the values of λ, all parameters were found to be significant in the sense of having at least 90% of the posterior mass away from zero.

The last observation time for a patient and whether they progressed at that time was captured by the tuple (T, E), as shown in FIG. 9. This quantity was modelled conditional on the vector comprising one or more patient's SLD measurements, denoted X, and non-target progression event time, N. T is set to the last observation time for a patient. E was determined conditional on X and N according to RECIST criteria. For example, when E=1, progression happened at T=1 if the patient had either target or non-target progression at time T. Non-target progression event occurred at time T when N=T by definition, while the relationship between target progression and X was slightly more complicated. Per RECIST, target progression occurred at time T when X_{T} was greater than 1.2 times the observed minimum of X up to time T and/or X_{T} was also at least 5 mm larger than the observed minimum. Furthermore, for all t<T, Xₜ was less than or equal to this threshold, the target progression did not occur earlier. his was expressed as the following inequalities holding:

$${X}_{T} > max \{ 1.2 {min}_{0 \leq s < T} {X}_{s} , 5 + {min}_{0 \leq s < T} {X}_{s} \}$$

$${X}_{t} \leq max \{ 1.2 {min}_{0 \leq s < t} {X}_{s} , 5 + {min}_{0 \leq s < t} {X}_{s} \} , ∀ t < T .$$

Similarly, E=0 when the patient dropped out at time T without progressing, or more specifically when N>T and all X, including up to time T, were less than the aforementioned threshold. Bayesian inference the data (T, E) was conditioned on, or held fixed, and values of the unknown quantities X and N that explain the fixed (T, E) were estimated via Bayes' rule and in this case Markov chain Monte Carlo (MCMC) sampling. To capture right-censoring in published studies, dropout was treated as a time-to-event that occurred at time T when E=0. It was modeled using a constant arm-specific hazard rate d.

Whether a patient experienced any response and whether they experienced complete response was captured by the tuple (O, C). As with the progression time and status, this quantity was also modeled conditionally on X and N. O was set to 1 if the minimum of Xₜ was less than 0.7R 0 and non-target progression event did not occur up to and including that time, otherwise O is set 0. C which denotes complete response was set to 1 if and the minimum of Xₜ was less than 2 mm. Although complete response was described in RECIST criteria as the complete disappearance of all lesions. This boundary was chosen for two reasons. First, numerically the minimum of Xₜ was a positive number and thus a practical threshold was chosen. Second, this was a small enough value in practice such that disappeared lesions was no longer be spotted in imaging.

The entire generative process of the data, for example, the joint distributional structure, was specified in FIG. 9 and summarized by the following joint distribution over data and parameters:

p(R₀,k_{g},f,kₖₖᵢₗₗ,kₖₖᵢₗₗᵣ|μ_{R}_{₀},τ_{R}_{₀},μₖ_{_{g}},τₖ_{_{g}},μ_{f},τ_{f},kₖᵢₗₗ,kₖᵢₗₗᵣ)·p(X|R₀,k_{g},f,kₖₖᵢₗₗ,kₖₖᵢₗₗᵣ,σ)p(N|R₀,k_{g},kₖₖᵢₗₗ,kₖₖᵢₗₗᵣ,λ)·p(T,E|X,N)p(O,C|X,N).

In FIG. 9, the bottom left plot shows that individual-specific parameters have great impact on the latent tumor dynamics. The bottom middle plot shows the latent X, N observations relate to the actual latent SLD observations. The bottom right table shows that the set of random variables T, E, O, C relate to the actual published PFS and ORR.

### Bayesian Inference of Model Parameters Given Observed Data

Inference of model parameters was done using standard Bayesian inversion of the data generating process facilitated by MCMC posterior sampling. Specifically, priors on model parameters were specified and multiplied by the conditional distribution above to obtain a joint distribution over model parameters and observed data. Observed data was then fixed to observed values, for example, conditioned on, and the resulting unnormalized posterior density of the model parameters was sampled using MCMC.

For most parameters, priors were set to allow for a large range of plausible values that allowed the model to explain the data on a variety of examples from published studies show in the results section, while also decreasing the possibility of unrealistic and numerically unstable values. Specifically, some parameters were μₖ_{_{g}}˜HalfNormal(0,0.1) and kₖₖᵢₗₗ˜HalfNormal(0,0.02). The latter placed prior mass on reasonable values of kₖₖᵢₗₗ while avoiding fast killing rates that led to numerical instability in the integration of the hazard. Meanwhile kₖₖᵢₗₗᵣ˜Exponential(1000) served two purposes. First, it placed a significant amount of prior mass on zero which encouraged the growth dynamics of the more resistant cells in control/placebo arms to be determined entirely by k_{g}. This helped with identifiability issues without having to explicitly set kₖₖᵢₗₗᵣ in the control arm to zero. Second, because the exponential distribution had a heavy tail, it allowed for ample flexibility in practice to better capture the long-term benefit a treatment might have over a control or placebo, since the faster killing rate, kₛ, mostly captured early response rather than longer term progression behavior. For μ_{f}, a fixed value of 0, corresponding to f=0.5, was tried. However, it was found that the more relaxed μ_{f}˜N(0,0.5) provided better fits in practice while still encouraging the SLD trajectories to maintain the characteristic biexponential U-shape commonly seen in practice. Priors for the variance parameters were set to τₖ_{_{g}}˜HalfNormal(0,1) and τ_{f}˜HalfNormal(0,2) respectively. This avoided extremely large and numerically unstable values of interindividual variability while allowing for enough flexibility to fit observed data and in particular the observed number of complete responses seen in certain published studies. μ_{R}_{₀} and τ_{R}_{₀} were set to fixed values that allowed for reasonable values of R₀ based on distributions R₀ seen on internal data and the number of patient reported in the published study to be measurable, for example, had an initial R₀ greater than 10 mm. For studies in the examples section, μ_{R}_{₀}=10 and τ_{R}_{₀}=0.5 since this led to about half the patients being measurable which was around the number generally seen in the published studies used in the results section.

Observed data (e.g., PFS data) was extracted from published studies that included PFS curves and risk tables, ORR, and the number of complete responders to incorporate as much information into the model as possible. Risk tables and PFS curves were combined using the standard formula for the Kaplan-Meier estimate to obtain at each time, τ, in the risk table, the number of patients who progressed and dropped out at that time, M_{τ1} and M_{τ0} respectively. The likelihood of the progression data was then set to according equation below:

·p(T,E|X,N)=Π_{τ}[Πᵢ₌₁^{M}^{_{τ1}}p(T_{τ1i}=τ,E_{τ1i}=1|X_{τ1i},N_{τ1i})Πᵢ₌₁^{M}^{_{τ0}}p(T_{τ0i}=τ,E_{τ0i}=0|X_{τ0i},N_{τ0i})]

where the triple subscript denoted the ith patient in the group with last observation time τ and either progression or no progression respectively. Because T and E were determined from X and N via the RECIST rules, when the former was set to a fixed value, this informed the possible values that the latter may take on which in turn informs the individual dynamics parameters and so on up the data-generating chain. Implementation-wise, the individual likelihood term p(T_{τ·i}=τ,E_{τ·i}=·|X_{τ·i},N_{τ·i}) was not a literal expression included in the joint probability density in Stan. Rather, it was a set of constrains on X_{τEi} and N_{τEi} that were implemented as inequality constraints on those specific parameters in Stan. As a simple example, if T was equal to the first post-baseline scan time and E=1 then X₁ was constrained to be greater than both 1.2R₀ and 5+R₀ or N=1.

Information about the number of total and complete responders (e.g., patients who responded to treatment) was incorporated into the likelihood slightly differently. Published studies generally specified the number of total and complete responders, but did not specify which patients were the responders, thus making the value of O_{τ˜i} and C_{τ·i} unknown. The flexibility of a probabilistic programming language like Stan nonetheless allowed for the conditioning of this information in the joint probability density. Denoting the observed number of total and complete responders as Ō_{obs} and C̅_{obs} respectively, the information was incorporated into the model using the following probabilistic statements

Ō_{obs}˜N(ΣO_{τEi},σ_{O})

C̅_{obs}˜N(ΣC_{τEi},σ_{C})

where the sums were taken over the response statuses of all patients and 6 terms were auxiliary hyperparameters set to small values to keep the values of the sum of these quantities in the model close to their observed values. Typically these were set to 1.

### Simulated Data

To demonstrate the model's ability to recover tumor dynamics using PFS and ORR information, a simulated dataset with known ground truth parameter values was simulated and inferred back. Population-level growth dynamics parameters shared between arms were set to μₖ_{_{g}}=0.01, τₖ_{_{g}}=0.8, μ_{f}=0.5, τ_{f}=2. Two arms representing a control arm and a treatment arm were simulated with 100 patients each with arm-specific killing parameters k_{kill-ctrl}=0.01, k_{killr-ctrl}=0 and k_{kill-trt,}=0.02, kₖᵢₗₗᵣ=0.003 respectively. Scans were taken every two months. As is the case with real data, the model used the information from the resulting risk table and the number of responders.

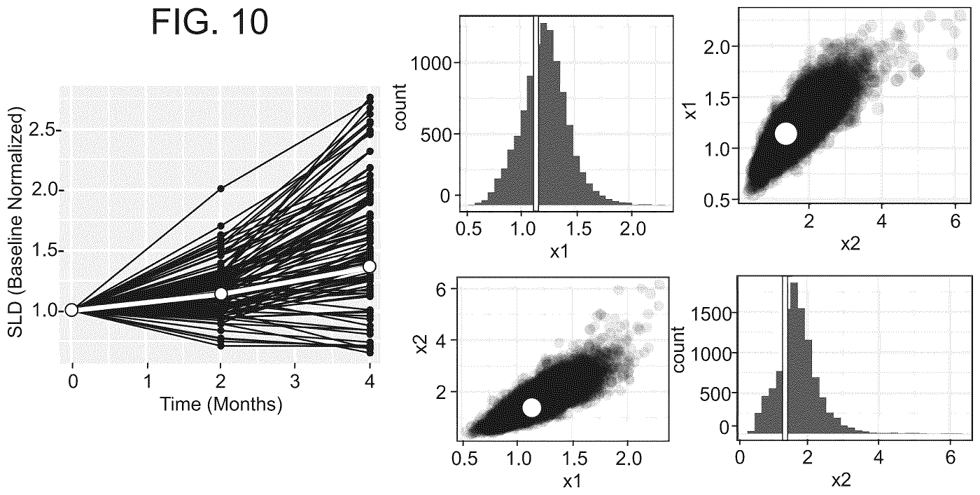

FIG. 10 shows several posterior draws of SLD (e.g., X) for a single patient from the control arm who progressed at the second post-baseline scan with the ground truth values overlayed in red. Conditioning on this information amounts to imposing constraints on X, and yields posterior draws from the space of possible SLD values that could have plausibly led to the patient progressing at the second post-baseline scan. X had a joint posterior distribution that was also dependent on other parameters such as R₀ and N which were not depicted here. For example, several draws showed a declining SLD over time, but this did not contradict the fact that this patient had progression, as in these cases the patient's progression was explained by non-target progression.

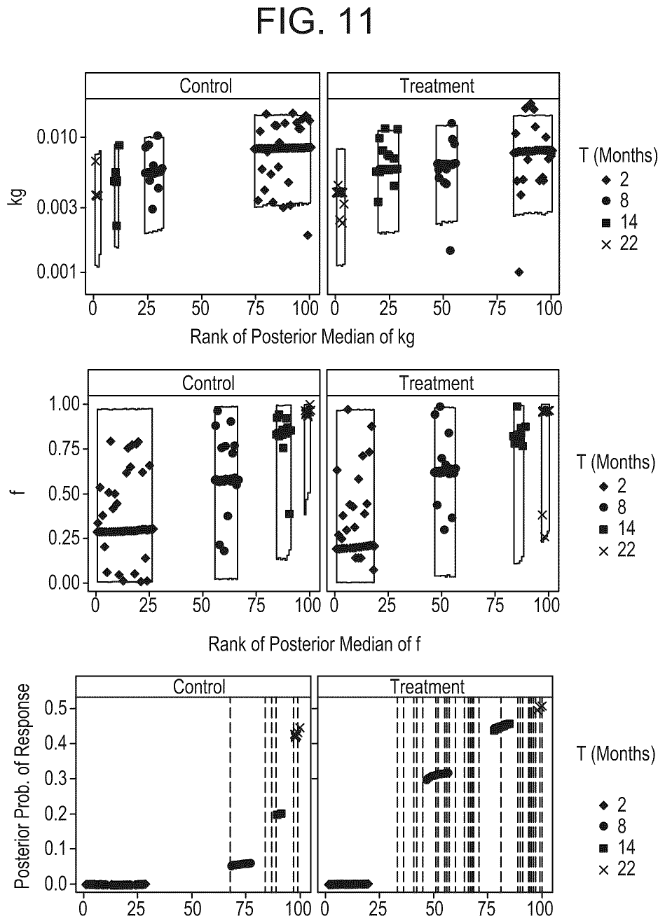

FIG. 11 summarizes for each patient their marginal posterior values estimated from the model along with ground truth values. The top and middle subfigures showed the marginal posterior median and 90% credible intervals for each patient's k_{g} and f parameters. Ground truth values were overlayed showing good posterior coverage of the true parameter values. Estimates for each patient were shown by the patient's progression or dropout time, T, revealing two patterns of note. First, the later the known progression time of a patient occurs, the more posterior weight was given to larger values of k_{g} and f as would be expected. Second, since the model could estimate the patients' progression or dropout time T, and whether they actually progressed at that time E, and not their actual SLD measurements, patients who had identical values of T and E had identical posterior distributions for their individual parameters. The bottom two subfigures in FIG. 11 shows the marginal posterior probabilities of each individual patient being one of the responders, with the actual ground truth responders being depicted with a dotted line. As described previously, the number of responders was known, not which patients were the responders, thus at each posterior sample a different combination of the patients formed the responder group. Intuitively, the patients who progress later had a higher chance of belonging to this group, and this was captured by the model in terms of their higher posterior probability.

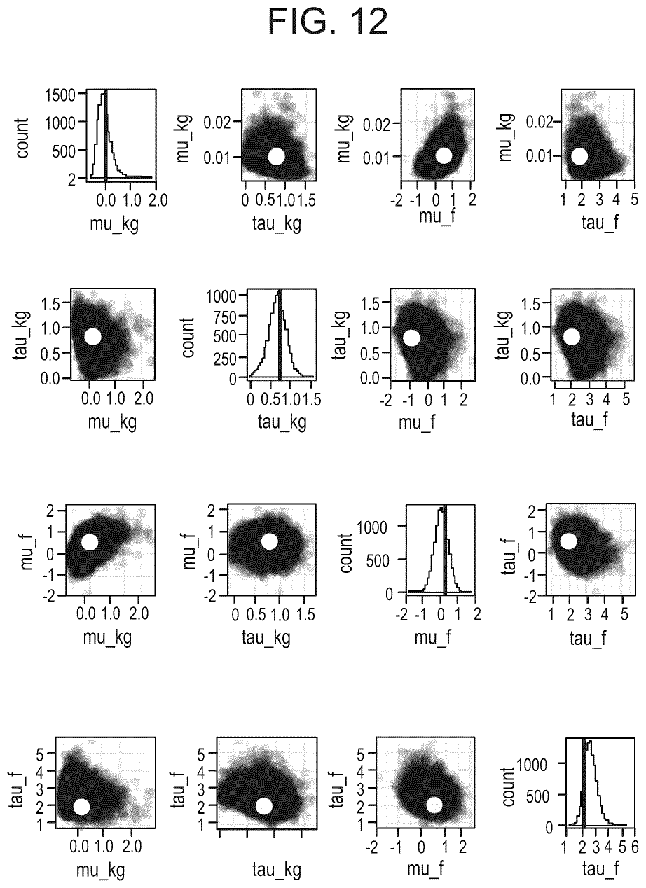

FIG. 12 shows posterior draws of the trial and arm-specific parameters with their ground truth values overlayed (note that the kₖᵢₗₗᵣ parameters were omitted due to space constraints). The posterior distributions showed good coverage of the ground truth values illustrating the model's ability to capture population-level dynamics using summary PFS and ORR information. The pairs plots also reflected the complicated nonlinear relationship between the dynamic parameters that was captured by the posterior. For example, μₖ_{_{g}} (as mu_kg in FIG. 12) and μ_{f} (as mu_f in FIG. 12) had correlated posterior distributions as higher values of f was offset by higher values of k_{g} in order to adequately describe the data.

### Published Studies

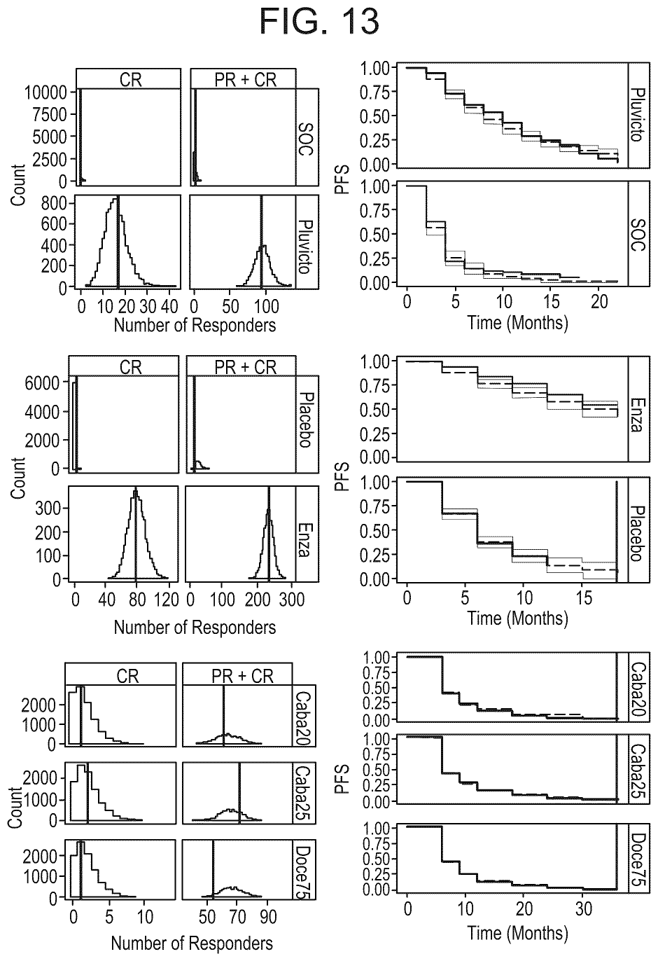

To illustrate the model's ability to capture tumor dynamic information from published studies, the method was applied to three published studies in metastatic, castration-resistant prostate cancer (mCRPC). These studies were summarized in Table 1. All models were fit until R-hat values for all parameters were below 1.05 as per standard guidance. To assess the fit of the model to the published data, posterior predictive checks (PPCs) were conducted where each arm of the trial was repeated with all new values of the individual-level parameters R₀, k_{g}, f drawn conditional on each posterior draw of the population-level parameters. The entire generative process of the data was then simulated once for each posterior draw to obtain a PFS curve and total number of responses for each draw. These simulated values were then overlayed with observed values in a PPC to assess the adequacy of the fits. The resulting plots were shown in FIG. 13. Although no ground-truth values were available for either population-level, individual-level, or SLD parameters, the model showed a good ability to capture the observed PFS curves and response data across the various published studies.

TABLE 1

| | | | Inclusion | | | Median | PR + | |
|---|---|---|---|---|---|---|---|---|
| Name | Year | Phase | Criteria | Arm | N | PFS | CR | CR |
| VISION | 2021 | 3 | Post- | SOC | 196 | 8.7 | 64 | 0 |
| | | | chemo | Pluvicto | 385 | 3.4 | 184 | 17 |
| PREVAIL | 2014 | 3 | Chemo- | Placebo | 801 | 3.9 | 19 | 4 |
| | | | naive | Enza | 832 | NA | 233 | 78 |
| FIRSTANA | 2017 | 3 | Chemo- | Caba20 | 389 | 4.4 | 61 | 1 |
| | | | naive | Caba25 | 388 | 5.1 | 72 | 2 |
| | | | | Doce75 | 391 | 5.3 | 54 | 1 |

### Simulating Novel Trial Conditions Using Published Studies

Posterior estimates from two different published studies from the previous subsection were combined in an in silico trial to 1) assess the efficacy of a drug treatment that was tested on post-chemo patients in a chemo-naïve setting; 2) assess the efficacy of a combination of one or more drug treatments; 3) compare the head-to-head results of each drug treatment as a monotherapy and as a combination of one or more drug treatments.

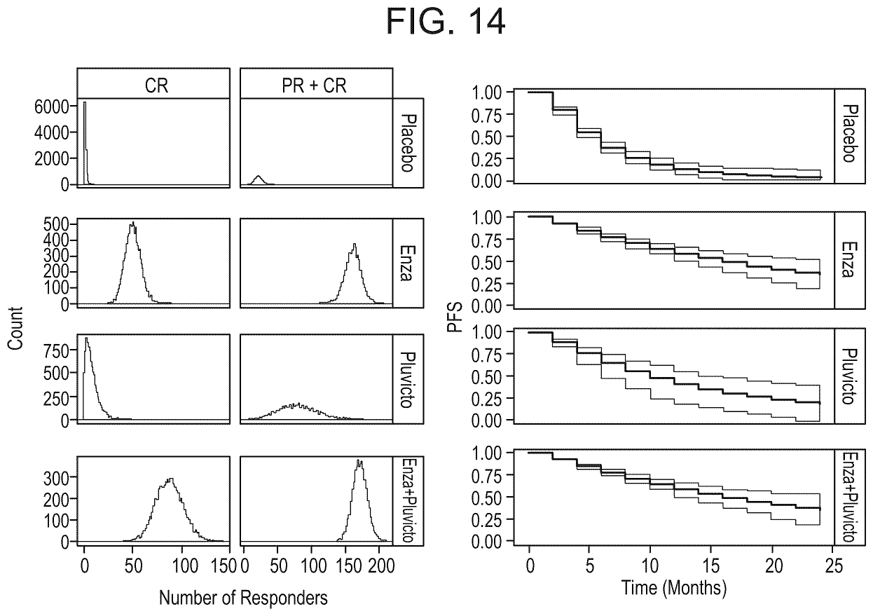

Four arms with 500 patients each were simulated in a chemo-naïve setting by taking estimates of the μₖ_{_{g}}, τₖ_{_{g}}, μ_{f}, τ_{f} parameters from the PREVAIL study from the previous subsection that was conducted in a chemo-naïve setting (e.g., a patient has not been treated with chemotherapy). A placebo and Enza arm were simulated using estimated kₖₖᵢₗₗ parameters from the respective arms of the same model fit. A Pluvicto® arm was simulated by using the kₖₖᵢₗₗ parameters fit to the VISION study in the previous subsection. Finally, the fourth arm which was assigned the combination therapy of both Enza and Pluvicto® was simulated by assigning this arm a kill rate that was the sum of the kill rates from the Enza and Pluvicto® fits from the two respective models. The additive killing was a reasonable first-pass assumption given that these therapies work via different mechanisms. To account for the estimation uncertainty of these parameters, as well as other sources of variation such as trial-to-trial variation, a simulation was conducted for each posterior draw of the trial and arm-specific parameters. FIG. 14 shows the simulated number of responses and PFS curves for the four arms of the simulated trial. The Enza arm seemingly performed better than the Pluvicto arm in terms of PFS, PR+CR, and CR while the combination seems to outperform the Enza arm in terms of CR. The simulations were used to provide more formal probability estimates. Specifically, the Enza arm outperformed the Pluvicto arm in PR+CR, CR, and median PFS with probabilities 0.997, 0.999, and 0.898, respectively, while the combination arm outperformed the Enza-only arm in the same three categories with respective probabilities 0.730, 0.995, and 0.437.

A Bayesian generative model that ties together tumor dynamics with published PFS and response data was introduced. The model allowed for estimation of important tumor dynamic parameters and information using published data. Several results were shown. First, an example using simulated data where the ground truth was known was shown to illustrate the model's ability to recover tumor dynamic parameters using published data. Second, the model was applied and shown to have a good fit to three published studies from real mCRPC trials. Last, these estimated parameter values were combined to compare these therapies in an in silico trial under a novel trial setting as well as in combination with one another.

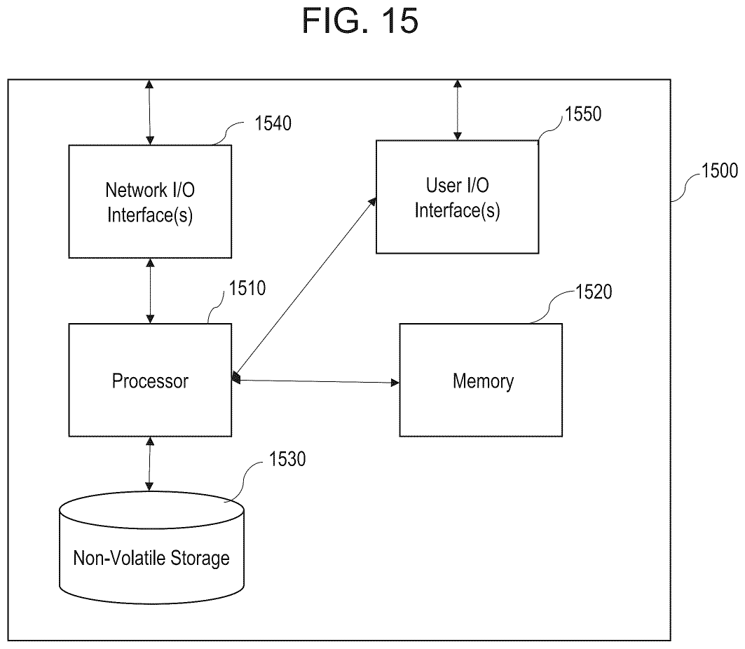

An illustrative implementation of a computer system 1500 that may be used in connection with any of the embodiments of the technology described herein is shown in FIG. 15. The computer system 1500 includes one or more processors 1510 and one or more articles of manufacture that comprise non-transitory computer-readable storage media (e.g., memory 1520 and one or more non-volatile storage media 1530). The processor 1510 may control writing data to and reading data from the memory 1520 and the non-volatile storage device media 1530 in any suitable manner, as the aspects of the technology described herein are not limited to any particular techniques for writing or reading data. To perform any of the functionality described herein, the processor 1510 may execute one or more processor-executable instructions stored in one or more non-transitory computer-readable storage media (e.g., the memory 1520), which may serve as non-transitory computer-readable storage media storing processor-executable instructions for execution by the processor 1510.

Computer system 1500 may also include a network input/output (I/O) interface 1540 via which the computing device may communicate with other computing devices (e.g., over a network), and may also include one or more user I/O interfaces 1550, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

As used herein, the singular terms “a,” “an,” and “the” may include plural referents, unless the context clearly dictates otherwise.

As used herein, the terms “connect,” “connected,” and “connection” refer to an operational coupling or linking. Connected components can be directly or indirectly coupled to one another, for example, through another set of components.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein, the terms “approximately,” “substantially,” “substantial” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, two numerical values can be deemed to be “substantially” the same if a difference between the values is less than or equal to ±10% of an average of the values, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the present disclosure has been described and illustrated with reference to specific embodiments thereof, these descriptions and illustrations do not limit the present disclosure. It should be understood by those skilled in the art that various changes may be made and equivalents may be substituted without departing from the true spirit and scope of the present disclosure as defined by the appended claims. The illustrations may not be necessarily drawn to scale. There may be distinctions between the artistic renditions in the present disclosure and the actual apparatus due to manufacturing processes, tolerances and/or other reasons. There may be other embodiments of the present disclosure which are not specifically illustrated. The specification (other than the claims) and drawings are to be regarded as illustrative rather than restrictive. Modifications may be made to adapt a particular situation, material, composition of matter, technique, or process to the objective, spirit and scope of the present disclosure. All such modifications are intended to be within the scope of the claims appended hereto. While the techniques disclosed herein have been described with reference to particular operations performed in a particular order, it will be understood that these operations may be combined, sub-divided, or re-ordered to form an equivalent technique without departing from the teachings of the present disclosure. Accordingly, unless specifically indicated herein, the order and grouping of the operations are not limitations of the present disclosure.

1. A computer-implemented method for estimating tumor growth, the method comprising:
obtaining, by one or more processors, progression-free survival (PFS) data for a plurality of patients, the PFS data indicating (i) a plurality of observation times, and (ii) how many of the plurality of patients, at each of the plurality of observation times, had a PFS event within a most recent time window;
determining, by the one or more processors and based on the PFS data, a population distribution for one or more patient-specific parameters;
obtaining, by the one or more processors, measured tumor growth data for a particular patient subject to a drug treatment;
estimating, by the one or more processors, tumor growth for the particular patient based on (i) the measured tumor growth data and (ii) the population distribution for the one or more patient-specific parameters; and
causing, by the one or more processors, a display to present a visual indication of the estimated tumor growth for the particular patient.

2. The computer-implemented method of claim 1, wherein determining the population distribution for the one or more patient-specific parameters comprises determining a growth curve function comprising the one or more patient-specific parameters.

3. The computer-implemented method of claim 2, wherein the growth curve function comprises at least one of an exponential growth function, a logistic growth function, or an ordinary differential function.

4. The computer-implemented method of claim 1, wherein the one or more patient-specific parameters comprise a parameter for baseline-normalized sum-of-longest diameters (SLD) measurement.

5. The computer-implemented method of claim 1, wherein the one or more patient-specific parameters comprise a parameter for growth rate.

6. The computer-implemented method of claims 5, wherein the parameter for growth rate comprises a parameter for baseline growth rate without treatment.

7. The computer-implemented method of claim 1, wherein the one or more patient-specific parameters comprise a parameter for a proportion of drug-sensitive tumor cells in a patient of the plurality of patients.

8-9. (canceled)

10. The computer-implemented method of claim 2, wherein determining the population distribution for the one or more patient-specific parameters includes fitting the growth curve function to observations at the plurality of observation times.

11. (canceled)

12. The computer-implemented method of claim 1, wherein estimating tumor growth for the particular patient includes:
modeling a tumor growth rate for the particular patient using a pharmacokinetic-pharmacodynamic (PKPD) model having a first term representing tumor size variation in the particular patient without the drug treatment and a second term representing a contribution to tumor size variation in the particular patient due to the drug treatment; and
jointly estimating one or more parameters of the first term and one or more parameters of the second term by fitting the PKPD model to the measured tumor growth data, in part by using the population distribution for the one or more patient-specific parameters to set constraints for the one or more parameters of the first term.

13. The computer-implemented method of claim 12, wherein the one or more parameters of the second term include one or more of:
a drug concentration in plasma of the particular patient;
a max kill rate for the particular patient; and
half-maximal effective concentration (EC50).

14. The computer-implemented method of claim 1, wherein estimating the tumor growth further comprises obtaining an overall response rate for the particular patient subject to the drug treatment.

15. The computer-implemented method of claim 1, wherein estimating the tumor growth further comprises obtaining one or more nontarget events for the particular patient subject to the drug treatment.

16. The computer-implemented method of claim 10, wherein the observations include, for each patient of the plurality of patients, a first observation at a first time indicating that the patient did not have a PFS event before the first time, and a second observation at a second time indicating that the patient had a PFS event before the second time.

17. The computer-implemented method of claim 1, wherein:
the PFS data corresponds to a specific cancer type; and
estimating tumor growth for the particular patient includes estimating tumor growth for a patient diagnosed with the specific cancer type.

18. The computer-implemented method of claim 1, wherein causing the display to present the visual indication of the estimated tumor growth for the particular patient includes causing the display to show a trajectory of tumor growth for the particular patient if the particular patient had not had the drug treatment.

19. (canceled)

20. The computer-implemented method of claim 1, wherein the PFS data comprises at least one of a digitized PFS plot or a PFS risk table.

21. The computer-implemented method of claim 1, wherein the PFS data indicates how many of the plurality of patients, at each of the plurality of observation times and within the most recent time window, had a baseline-normalized sum-of-longest diameters (SLD) measurement of at least 1.2 or had new lesions appear.

22. (canceled)

23. The computer-implemented method of claim 1, further comprising, adjusting a dose of the drug treatment based on the estimated tumor growth for the particular patient.

24. A computer system for estimating tumor growth, the computer system comprising:
a data storage device storing processor-readable instructions; and
a processor configured to execute the instructions to perform a method including:
obtaining progression-free survival (PFS) data for a plurality of patients, the PFS data indicating (i) a plurality of observation times, and (ii) how many of the plurality of patients, at each of the plurality of observation times, had a PFS event within a most recent time window;
determining, based on the PFS data, a population distribution for one or more patient-specific parameters;
obtaining measured tumor growth data for a particular patient subject to a drug treatment;
estimating tumor growth for the particular patient based on (i) the measured tumor growth data and (ii) the population distribution for the one or more patient-specific parameters; and
causing a display to present a visual indication of the estimated tumor growth for the particular patient.

25-46. (canceled)

47. A non-transitory computer-readable medium containing instructions for estimating tumor growth that, when executed by a processor, cause the processor to perform a method comprising:
obtaining progression-free survival (PFS) data for a plurality of patients, the PFS data indicating (i) a plurality of observation times, and (ii) how many of the plurality of patients, at each of the plurality of observation times, had a PFS event within a most recent time window;
determining, based on the PFS data, a population distribution for one or more patient-specific parameters;
obtaining measured tumor growth data for a particular patient subject to a drug treatment;
estimating tumor growth for the particular patient based on (i) the measured tumor growth data and (ii) the population distribution for the one or more patient-specific parameters; and
causing a display to present a visual indication of the estimated tumor growth for the particular patient.

48-69. (canceled)
